(19) United States
(12) Patent Application Publication
(10) Pub. No.: US 2026/0254275 A1
(43) Pub. Date: Aug. 27, 2026

(54) External Charger for an Implantable Medical Device Employing Use of a Phase Angle Between Sense Coils

(71) Applicant: Boston Scientific Neuromodulation Corporation, Valencia, CA (US)

(72) Inventors: Tom Stouffer, Chatsworth, CA (US); Daniel Aghassian, La Canada, CA (US); Gaurav Gupta, Valencia, CA (US)

(21) Appl. No.: 19/386,979

(22) Filed: Nov. 12, 2025

Related U.S. Application Data
(60) Provisional application No. 63/764,350, filed on Feb. 27, 2025.

(51) Int. Cl. H02J 50/12; A61N 1/378; H02J 50/00; H02J 50/90

(52) U.S. Cl. CPC H02J 50/12; A61N 1/3787; H02J 50/005; H02J 50/90

(57) ABSTRACT

An external charger for an Implantable Medical Device (IMD) is disclosed having a primary charging coil and preferably two sense coils. The sense coils provide feedback in the form of various monitored sense coil parameters that allow the charger to take various actions to monitor and regulate charging of the IMD, including determining an alignment between the charger and the IMD, adjusting the frequency of the magnetic field produced by the primary charging coil, adjusting a power of the magnetic field, and reception of back telemetry from the IMD. The charger further includes functions to set the power of the magnetic field in accordance with a sense coil parameter. These functions are selectable by a user to set a power mode for the charger.

### CROSS REFERENCE TO RELATED CASES

This is a non-provisional application of U.S. Provisional Patent Application Serial No. 63/764,350, filed February 27, 2025, which is incorporated herein by reference in its entirety, and to which priority is hereby claimed.

### FIELD OF THE INVENTION

The present invention relates to wireless external chargers for use in charging implantable medical devices.

### INTRODUCTION

Implantable stimulation devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Patent 6,516,227. However, the present invention may find applicability in any implantable medical device system.

As shown in FIGS. 1A-1C, an SCS system typically includes an Implantable Pulse Generator (IPG) 10 (Implantable Medical Device (IMD) 10 more generally), which includes a biocompatible device case 12 formed of a conductive material such as titanium for example. The case 12 typically holds the circuitry and battery 14 (FIG. 1C) necessary for the IMD 10 to function, although IMDs can also be powered via external RF energy and without a battery. The IMD 10 is coupled to electrodes 16 via one or more electrode leads 18, such that the electrodes 16 form an electrode array 20. The electrodes 16 are carried on a flexible body 22, which also houses the individual signal wires 24 coupled to each electrode. In the illustrated embodiment, there are eight electrodes (Ex) on each lead 18, although the number of leads and electrodes is application specific and therefore can vary. The leads 18 couple to the IMD 10 using lead connectors 26, which are fixed in a non-conductive header material 28, which can comprise an epoxy for example. The conductive case 12 may also act as an electrode.

In an SCS application, the electrode leads 18 are typically implanted in the spinal column inside the patient’s vertebrae and proximate to the dura in a patient’s spinal cord, preferably spanning left and right of the patient’s spinal column. Proximal contacts on the leads 18 are tunneled through the patient’s tissue to a distant location such as the upper buttocks where the IMD case 12 is implanted, at which point they are coupled to the lead connectors 26. SCS therapy is traditionally used to relieve symptoms such as chronic back pain. IMD 10 as described should be understood as including non-implantable External Trial Stimulators (ETSs), which mimic operation of the IMD 10 during trials periods when electrode array 20 has been implanted in the patient but the IMD has not. See, e.g., USP 9,259,574 (disclosing an ETS).

FIGS. 1B and 1C show plan and cross-sectional views of the IMD 10, with the case 12 removed in FIG. 1B for easier viewing of internal components. As shown, the IMD 10 typically includes a printed circuit board (PCB) 30, along with various electronic components 32 mounted to the PCB 30, some of which are shown in FIG. 3. Two coils (more generally, antennas) are shown in the IMD 10: a telemetry coil 34 used to transmit/receive data to/from an external controller (not shown); and a charging coil 36 for charging or recharging the IMD’s battery 14 using an external charger. These coils 34 operate on a principle of magnetic induction to both receive power from an external charger and to communicate bidirectionally with an external controller (not shown; see e.g. USP 11,559,693). Although not shown, the IMD 10 can also include a short-range RF antenna in lieu of telemetry coil 34 which can communicate with the external controller or the external charger using far-field electromagnetic waves, using the well-known Bluetooth standard for example.

FIG. 2A shows a plan view of a traditional external charger 50 for the IMD 10, while FIG. 2B shows the external charger 50 and IMD 10 in cross section with the external charger 50 wirelessly conveying power via a magnetic field 45 to the IMD 10, which power can be used to operate the IMD and/or recharge the IMD’s battery 14. The external charger 50 contains one or more PCB 54 on which electronic components 56 are placed, see USP 9,002,445, some of which are shown in FIG. 3. A user interface 58 including an on/off switch 60 allows a patient or clinician to operate the external charger 50 to start and stop generation of the magnetic field 45. User interface 58 may also include a Light Emitting Diode (LEDs) or other lamps and possibly also a speaker to indicate status. A battery 64 provides power for the external charger 50, which battery 64 may itself be rechargeable. The external charger 50 can also receive AC power from a wall plug for power, or to charge the battery 64. The charger 50 may also include a temperature sensor 68 to monitor the temperature during charging. If the temperature gets too hot (above a threshold), thus risking patent discomfort or injury, the charger 50 can be controlled to temporarily suspend producing the magnetic field 45 until the temperature drops to a safe level. In this regard, operation of the charger may duty cycle on and off, unbeknownst to the user. A hand-holdable housing 66 sized to fit a user’s hand contains all of the charger’s components.

Power transmission from the external charger 50 to the IMD 10 occurs wirelessly and transcutaneously through a patient’s tissue 25 via inductive coupling, and FIG. 3 shows details of the circuitry used to implement such functionality. Primary charging coil 52 in the external charger 50 is energized via charging circuit 70 with an AC current, Icharge, to create the AC magnetic field 45. This magnetic field 45 induces a current in the secondary charging coil 36 within the IMD 10, providing a voltage across coil 36 that is rectified (38) to DC levels and used to recharge the battery 14, either by application of a battery charging current Ibat or by application of a voltage to the battery. This voltage or current can be regulated by charging and protection circuitry 40 as shown. The frequency of the magnetic field 45 (and AC current Icharge) can be perhaps 80 kHz or so. When charging the battery 14 in this manner, it is typical that the housing 66 of the external charger 50 touches the patient’s tissue 25, perhaps with a charger holding device or the patient’s clothing intervening, although this is not strictly necessary. The charger 50 and IMD 10 can respectively include control circuitries 72 and 42 to control operation of charging and other functions in these devices.

The IMD 10 can send telemetry to the charger 50, and in the example shown in FIG. 3 this occurs using Load Shift Keying (LSK). LSK can generally be used to send any sort of digital data (LSK data) to the charger 50, including data that can be used to control charger 50 functionality. For example, during a charging session, the IMD 10 could LSK telemeter the temperature of the IMD 10 (as sensed by a temperature sensor in the IMD 10), the current voltage of the IMD 10’s battery 14 (Vbat, which will increase as the battery charges), or the charging current Ibat that battery is receiving, all of which may be relevant to adjusting the power of the magnetic field 45 or turning it off.

In the example shown, LSK data telemetered from the IMD 10 is limited to transmission of an end-of-charge (EoC) signal, which informs the charger 50 to stop producing the magnetic field 45. The EoC signal can be sent in a number of different circumstances. Primarily, the EoC signal is transmitted when the IMD 10 determines that its battery 14 is full, and therefore that charging is no longer needed. This determination can be made by comparing the battery voltage, Vbat, to a threshold in the IMD’s control circuitry 42. However, the EoC signal can also be sent for example if the IMD’s temperature (T, as sensed by an IMD temperature sensor) is too high (again, above a threshold) during a charging session.

An LSK modulator 74 in the IMD 10 is enabled when the EoC signal (or other LSK data) is to be transmitted. As shown, the LSK modulator 74 may comprise a firmware module in the IMD’s control circuitry 42. The LSK modulator 74 outputs a series of digital data bits each with a bit duration td. While this series of bits could be more random (e.g., if more detailed LSK data like Ibat, Vbat, or T is being sent), in the depicted example the EoC signal comprises a sequence of a specific number (e.g., 128) of alternating bits (010101…). This signal is sent to the gates of switches 76, thus alternatively opening (0) and closing (1) them, which causes the ends of the secondary coil 36 in the IMD 10 to be either floating (as they are normally when receiving the magnetic field 45) or grounded.

Repeatedly grounding and floating the ends of the coil 36 via the EoC signal affects the mutual impedance between the charger 50 and the IMD 10, and in particular the mutual impedance between the primary charging coil 52 and the secondary charging coil 36. More specifically, this change in mutual impedance causes a reflected impedance which is detectable at the charger 50. Assuming that AC current Icharge through the primary charging coil 52 is of a constant magnitude, the reflected impedance will cause the magnitude of the AC voltage across this coil, Vcoil, to change, with this voltage being lower when the secondary coil 36 is floating (when EoC=0), and higher when that coil is grounded (when EoC=1).

Thus, this coil voltage Vcoil across the primary charging coil 52 can be assessed to recover the transmitted LSK data. This occurs by rectifying (78) the magnitude of Vcoil to a DC voltage, Vcoil(dc), and providing this voltage to a LSK demodulator 80. The LSK demodulator 80 monitors for changes in the magnitude of Vcoil(dc), and assesses whether such changes occur in accordance with the bit duration (td) and number of bits (e.g., 128) in the EoC signal. If the LSK demodulator 80 upon assessment of Vcoil(dc) detects receipt of the EoC signal, the LSK demodulator 80 informs the control circuitry 72 in the charger to cease producing Icharge through the primary charging coil 52, thus ceasing production of the magnetic field 45. Digital portions of the LSK demodulator 80 may be included as part of the charger’s control circuitry 72.

The IMD 10 may take other steps after transmitting an EoC signal. For example, after transmitting the EoC signal, the IMD 10 may decouple (e.g., ground via switches 76) its secondary coil 36 for some time to prohibit further receipt of the magnetic field 45.

### SUMMARY

An external charger for wirelessly providing energy to an implantable medical device (IMD) is disclosed, which may comprise: a charging coil configured when energized to produce a magnetic field to wirelessly provide energy to the IMD during a charging session; a first sense coil, wherein the first sense coil is configured to be induced by the magnetic field with a first induced signal; a second sense coil, wherein the second sense coil is configured to be induced by the magnetic field with a second induced signal; and control circuitry configured to determine a phase difference between the first and second induced signals.

The external charger can include any of the following modifications or additions in any combination. In one example, the control circuitry is further configured to determine an alignment between the charging coil and the IMD using the phase difference. In one example, the control circuitry is configured to determine the alignment by determining whether the charging coil and the IMD are aligned or misaligned using the phase difference. In one example, the control circuitry is configured to determine whether the charging coil and the IMD are aligned or misaligned by comparing the phase difference to a threshold. In one example, the control circuitry is configured to determine that the charging coil and the IMD are aligned when the phase difference is equal to or larger than the threshold, and to determine that the charging coil and the IMD are misaligned when the phase difference is less than the threshold. In one example, the external charger further comprises a user interface comprising one or more user interface elements, wherein when the control circuitry determines that the charging coil and the IMD are misaligned, the control circuitry is further configured to enable one or more of the user interface elements. In one example, the one or more user interface elements comprise one or more of a visual indicator or a speaker. In one example, the control circuitry is configured to determine the alignment between the charging coil and the IMD using the phase difference while producing the magnetic field with a constant power and frequency. In one example, the external charger further comprises a circuit board comprising the charging coil, the first sense coil, and the second sense coil. In one example, the charging coil comprises a wire winding affixed to a side of the circuit board. In one example, the first and second sense coils comprise one or more traces in the circuit board. In one example, the first sense coil, and the second sense coil are concentric. In one example, the first sense coil has a radius larger than a radius of the second sense coil. In one example, the charging coil has a radius larger than the radii of the first and second sense coils. In one example, the control circuitry is configured to determine the phase difference periodically during the charging session. In one example, the control circuitry is configured to use the phase difference to adjust a power of the magnetic field. In one example, the external charger comprises a first housing comprising the charging coil and the first and second sense coils, a second housing comprising the control circuitry, and a cable for passing signals between the first and second housings. In one example, the second housing further comprises an amplifier, wherein the amplifier is configured to energize the charging coil to produce the magnetic field. In one example, the second housing further comprises a user interface. In one example, the first housing comprises at least one temperature sensor.

A method for wirelessly providing energy to an implantable medical device (IMD) is disclosed, which may comprise: energizing a charging coil in an external charger to produce a magnetic field to wirelessly provide energy to the IMD during a charging session; inducing by the magnetic field a first induced signal on a first sense coil in the external charger; inducing by the magnetic field a second induced signal on a second sense coil in the external charger; and determining a phase difference between the first and second induced signals.

The method can include any of the following modifications or additions in any combination. In one example, the method further comprises determining an alignment between the charging coil and the IMD using the phase difference. In one example, the alignment is determined by determining whether the charging coil and the IMD are aligned or misaligned using the phase difference. In one example, alignment or misalignment is determined by comparing the phase difference to a threshold. In one example, alignment is determined when the phase difference is equal to or larger than the threshold, and wherein misalignment is determined when the phase difference is less than the threshold. In one example, the external charger comprises a user interface comprising one or more user interface elements, wherein determining misalignment enables one or more of the user interface elements. In one example, the one or more user interface elements comprise one or more of a visual indicator or a speaker. In one example, the alignment is determined using the phase difference while producing the magnetic field with a constant power and frequency. In one example, the external charger comprises a circuit board comprising the charging coil, the first sense coil, and the second sense coil. In one example, the charging coil comprises a wire winding affixed to a side of the circuit board. In one example, the first and second sense coils comprise one or more traces in the circuit board. In one example, the charging coil, the first sense coil, and the second sense coil are concentric. In one example, the first sense coil has a radius larger than a radius of the second sense coil. In one example, the charging coil has a radius larger than the radii of the first and second sense coils. In one example, the phase difference is determined periodically during the charging session. In one example, the method further comprises using the phase difference to adjust a power of the magnetic field. In one example, the external charger comprises a first housing comprising the charging coil and the first and second sense coils, a second housing, and a cable for passing signals between the first and second housings. In one example, the second housing further comprises an amplifier, wherein the amplifier energizes the charging coil to produce the magnetic field. In one example, the second housing further comprises a user interface. In one example, the first housing comprises at least one temperature sensor.

A system is disclosed, which may comprise: an implantable medical device (IMD) and an external charger for wirelessly providing energy to the IMD; the external charger comprising: a charging coil configured when energized to produce a magnetic field to wirelessly provide energy to the IMD during a charging session; a first sense coil, wherein the first sense coil is configured to be induced by the magnetic field with a first induced signal; a second sense coil, wherein the second sense coil is configured to be induced by the magnetic field with a second induced signal; and control circuitry configured to determine a phase difference between the first and second induced signals.

The system can include any of the following modifications or additions in any combination. In one example, the control circuitry is further configured to determine an alignment between the charging coil and the IMD using the phase difference. In one example, the control circuitry is configured to determine the alignment by determining whether the charging coil and the IMD are aligned or misaligned using the phase difference. In one example, the control circuitry is configured to determine whether the charging coil and the IMD are aligned or misaligned by comparing the phase difference to a threshold. In one example, the control circuitry is configured to determine that the charging coil and the IMD are aligned when the phase difference is equal to or larger than the threshold, and to determine that the charging coil and the IMD are misaligned when the phase difference is less than the threshold. In one example, the external charger further comprises a user interface comprising one or more user interface elements, wherein when the control circuitry determines that the charging coil and the IMD are misaligned, the control circuitry is further configured to enable one or more of the user interface elements. In one example, the one or more user interface elements comprise one or more of a visual indicator or a speaker. In one example, the control circuitry is configured to determine the alignment between the charging coil and the IMD using the phase difference while producing the magnetic field with a constant power and frequency. In one example, the external charger further comprises a circuit board comprising the charging coil, the first sense coil, and the second sense coil. In one example, the charging coil comprises a wire winding affixed to a side of the circuit board. In one example, the first and second sense coils comprise one or more traces in the circuit board. In one example, the charging coil, the first sense coil, and the second sense coil are concentric. In one example, the first sense coil has a radius larger than a radius of the second sense coil. In one example, the charging coil has a radius larger than the radii of the first and second sense coils. In one example, the control circuitry is configured to determine the phase difference periodically during the charging session. In one example, the control circuitry is configured to use the phase difference to adjust a power of the magnetic field. In one example, the external charger comprises a first housing comprising the charging coil and the first and second sense coils, a second housing comprising the control circuitry, and a cable for passing signals between the first and second housings. In one example, the second housing further comprises an amplifier, wherein the amplifier is configured to energize the charging coil to produce the magnetic field. In one example, the implantable medical device comprises an implantable neurostimulator device. In one example, the implantable medical device comprises an inflatable penile implant.

A computer readable medium is disclosed, which may comprise instructions executable in an external charger for wirelessly providing energy to an implantable medical device (IMD). The instructions when executed are configured to energize a charging coil in the external charger to produce a magnetic field to wirelessly provide energy to the IMD during a charging session; and determine a phase difference between a first induced signal induced on a first sense coil in the external charger and a second induced signal induced on a first sense coil in the external charger.

### BRIEF DESCRIPTION OF THE DRAWINGS

FIGS. 1A-1C show different views of an implantable pulse generator, a type of implantable medical device (IMD), in accordance with the prior art.

FIG. 2A shows an external charger in accordance with the prior art, and FIG. 2B shows this charger being used to charge a battery in an IMD.

FIG. 3 shows basic circuitry in both the external charger and the IMD, in accordance with the prior art.

FIGS. 4A and 4B show an improved external charger having an electronics module and charging coil assembly connected by a cable.

FIGS. 5A and 5B show top and bottom sides of a circuit board in the charging coil assembly, which circuit board includes a primary charging coil, and outer and inner sense coils.

FIG. 6 shows signaling between the charging coil assembly and the electronics module in the charger.

FIG. 7 shows the amplifier circuitry for driving the coil in the charging coil assembly, and further shows various firmware modules within the control circuitry of the charger.

FIGS. 8A-8D show various positionings between the charging coil assembly and the IMD being charged, some of which indicate poor coupling and poor alignment.

FIG. 9 shows the drive signal for driving the primary charging coil, and signal induced on the outer and inner sense coils, including the amplitude and phase angles of the induced signals.

FIGS. 10A and 10B show operation of a power algorithm programmed into a power module of the improved charger’s control circuitry, which allows for the selection of a plurality of different power modes to set a power of a magnetic charging field.

FIG. 11 shows operation of an alignment algorithm programmed into an alignment module of the improved charger’s control circuitry. The alignment algorithm as shown comprises an initial alignment detection algorithm and an alignment-while-charging algorithm.

FIG. 12 shows operation of the Load Shift Keying (LSK) demodulator in the charger, which in particular can be used to receive an End of Charge (EoC) signal telemetered from the IMD.

FIG. 13 shows an example of another type of implantable medical device able to be charged by the disclosed charger.

### DETAILED DESCRIPTION

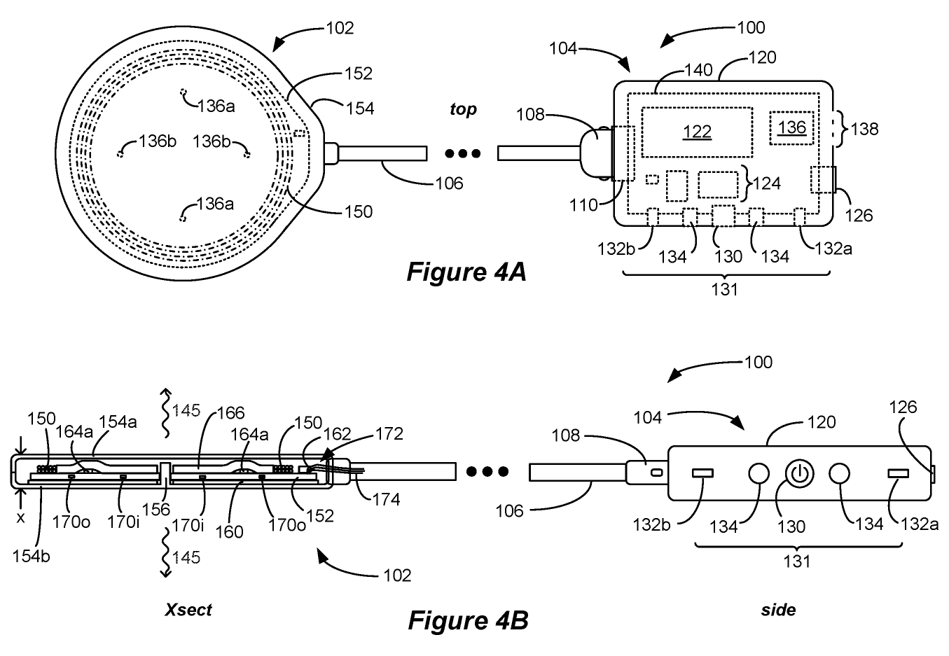

FIGS. 4A and 4B show a different design for an external charger 100 for an IMD 10, which is shown in a plan view (FIG. 4A) and in a side and cross-sectional view (FIG. 4B). The charger 100 is generally similar in structure to the charging system disclosed in U.S. Patent Application Publication 2017/0361113, and U.S. Provisional Patent Application Serial No. 63/764,364, filed February 27, 2025. These applications are incorporated herein by reference in their entireties. Despite similarities in their structures, the presently-disclosed design of charger 100 includes numerous functional improvements.

Charger 100 includes two main pieces: an electronics module 104 and a charging coil assembly 102 which includes a primary charging coil 150 used to produce a magnetic field 145 to charge the IMD 10’s battery 14, as explained earlier. The electronics module 104 and the charging coil assembly 102 are connected by a cable 106. The cable 106 may be separable from both the electronics module 104 and the charging coil assembly 102 via a port/connector arrangement, but as illustrated cable 106 is permanently affixed to the charging coil assembly 102. The other end of the cable 106 includes a connector 108 that can attach to and detach from a port 110 of the electronics module 104, although this end may be permanently affixed as well. The electronics module 104 preferably has a user interface 131, which is explained further below.

Separating the charging coil assembly 102 and the electronics module 104 via a cable 106 of significant length provides convenience for the patient, especially when compared to the single-housing design of charger 50 described earlier. For example, in a Spinal Cord Stimulation application where the IMD 10 is implanted behind the patient, the charging coil assembly 102 can be placed over the IMD 10 behind the patient, with the electronics module 104 looped around the patient’s waist and positioned in front of the patient where its user interface 131 can be more conveniently seen and accessed. Although not shown, a charging belt can be used to hold the charging coil assembly 102 and the electronics module 104 in convenient positions relative to the patient. This belt design can differ based upon the location at which the IMD 10 is implanted in the patient (e.g., Spinal Cord Stimulation v. Deep Brain Stimulation).

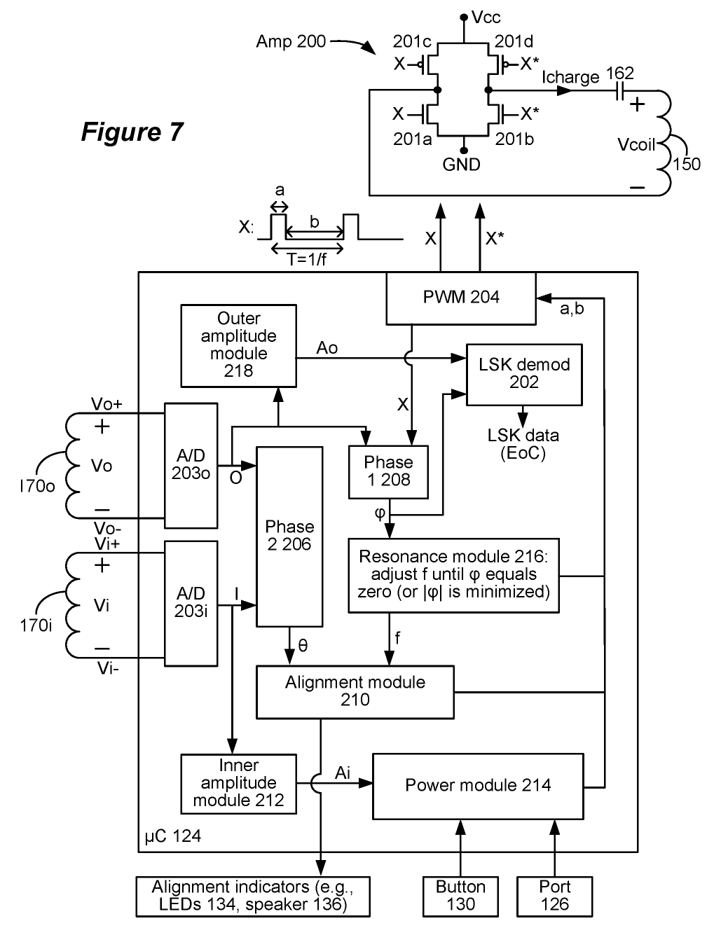

Electronics module 104 preferably includes within its housing 120 a battery 122 and control circuitry 124 needed for charging system operation. Control circuitry 124 is described in detail later with reference to FIG. 7, and can comprise a microcontroller programmed with firmware, such as any of the STM32L4 ARM series of microcontrollers provided by STMicroeletronics, Inc., as described at http:// www.st.com/ content/ st_com/ en/ products/ microcontrollers/ stm32-32-bit-arm-cortex-mcus/ stm32l4-series.html? querycriteria = productId=SS1580. Control circuitry 124 may also comprise an FPGA, DSP, or other similar digital logic devices. Control circuitry 124 can further comprise a memory programmed with firmware and accessible to a microcontroller or other digital logic device should that logic device not contain suitable on-chip memory. Control circuitry 124 may comprise a number of discrete components, and can comprise various analog circuitry and sensors in addition to digital processing logic. Electronics in the electronics module can be integrated via a circuit board 140. The electronics module 104 may further include a port 126 (e.g., a USB port) to allow its battery 122 to be recharged in conventional fashion, and/or to allow data to be read from or programmed into the electronics module, such as new operating software. Port 126 can also be used when setting a power mode for the charger 100, as explained further below.

Housing 120 may also carry user interface elements 131, as shown in the side view of FIG. 4B. These elements can include a button 130, and one or more visual indicators such as LEDs 132a, 132b, and 134. The button 130 can be used to start and stop generation of the magnetic field 145 from the primary charging coil 126, and can also be used to adjust the power mode. The button 130 may also be backlit by an LED. This LED could be lit for different purposes. For example, it could be lit to indicate that the charger 100 has been turned on and is producing a magnetic field 145. Alternatively, the LED associated with button 130 could be lit (e.g., green) to indicate good alignment between the charging coil assembly 102 and the IMD 10.

In one example, LED 132a is used to indicate the power status of the electronics module 104, i.e., the status (capacity) of the battery 122 and whether it needs charging. Charger battery LED 132a can for example be lit green, yellow, and red to indicate the (diminishing) capacity of the charger’s battery 122 based on measurements taken by the control circuitry 124 (such as the voltage of the battery 122), and can blink red when the charger battery 122 is critically low and recharging is required (via port 126).

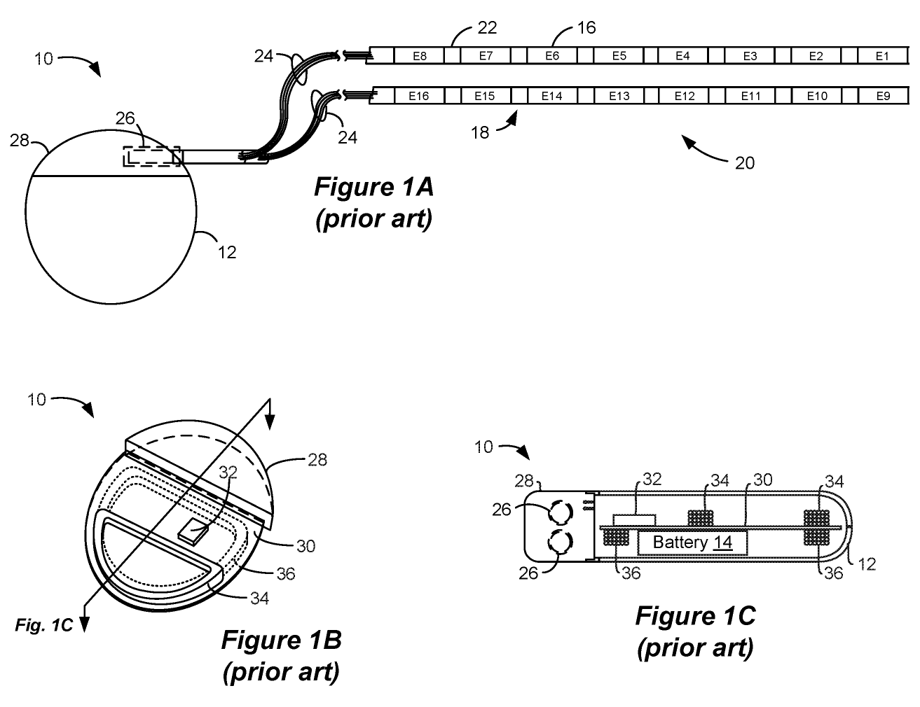

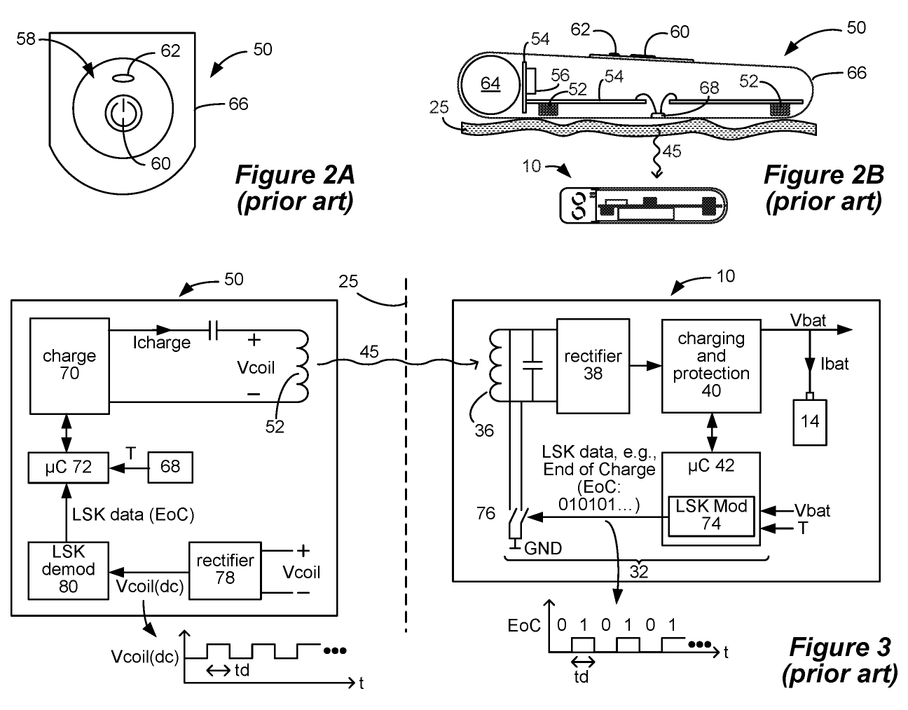

LED 132b may indicate the status of the battery 14 (FIG. 1C) in the IMD 10 being charged. In one example, implant battery LED 132b may not be controlled based on actual measurements of the capacity of the implant’s battery 14, but is instead controlled based on the charger 100’s operation. When the charger 100 is producing a magnetic field 145 and the charging coil assembly 102 is aligned with the IMD 10 being charged, the implant battery LED 132b can blink yellow to indicate that the implant battery 14 is being charged. Alignment between the charging coil assembly 102 and the IMD 10 is discussed further below. Once the implant’s battery 14 is full and the charger 100 receives telemetry from the IMD 10 of an end-of-charge (EoC) signal, this LED 132b can then be lit green. Alternatively, LED 132b may be controlled based on IMD battery 14 capacity measurements made in the IPG 10 (e.g., the voltage of battery 14). Such data can be telemetered from the IMD 10 to the charger 100, as discussed further below.

One or more LEDs 134 may be controlled in unison to indicate the degree of coupling/alignment between the charging coil assembly 102 and the IMD 10 during a charging session, as explained further below. For example, LEDs 134 may be lit amber to indicate poor alignment.

The charger 100 may also include a speaker 136 that emits tones or “beeps” in various circumstances. For example, the speaker 136 can emit beeps when the charging coil assembly 102 is misaligned with the IMD 10 being charged (in addition to also turning LEDs 134 amber). The speaker 136 may also issue a continuous double beep for a while (e.g., one minute) when the IMD’s battery 14 has been fully charged (once the charger receives the telemetered end-of-charge (EoC) signal). The speaker 136 may also beep a number of times when its battery 122 is critically low and thus the charger 100 is about to shut off (e.g., when LED 132a is red and blinking). The housing 120 may include openings comprising a speaker port 138 to facilitate sounds from the speaker 136 reaching the patient.

More complicated user interfaces, such as those incorporating a display, could also be provided with the electronics module 104, and as such, LEDs 132a, 132b, and 134 and button 130 could comprise indicators or touch-sensitive buttons on that display. User interface elements can be included on other faces of the electronic module’s housing 120, and may be placed such that they are easily viewed by the patient for the therapeutic application at hand (e.g., SCS, DBS).

Charging coil assembly 102 preferably contains only passive electronic components that are stimulated or read by the control circuitry 124 within the electronics module 104. Such components include the primary charging coil 150 already mentioned, which can comprise a winding of copper (e.g., Litz) wire that when energized by amplifier circuitry 200 (FIG. 7) in the electronics module 104 will create the magnetic field 145 that provides power to the IMD 10. As shown in FIG. 4B, the primary charging coil 150 is mounted to the top side of a circuit board 152 within a housing 154 of the charging coil assembly 102. Housing 154 is preferably formed of a plastic material (e.g., polycarbonate) and as shown preferably comprises a top housing portion 154a and a bottom housing portion 154b, which may be joined during manufacturing by screwing, snap fitting, ultrasonic welding, or solvent bonding. In one example, the charging coil assembly 102 contains no user interface elements, these instead being associated with the electronics module 104 as already described. However, the housing 154 of the charging coil assembly 102 may also have user interface elements (e.g., LEDs) to indicate statuses similar to those described with respect to LEDS 132a, 132b, and 134, or other indicators.

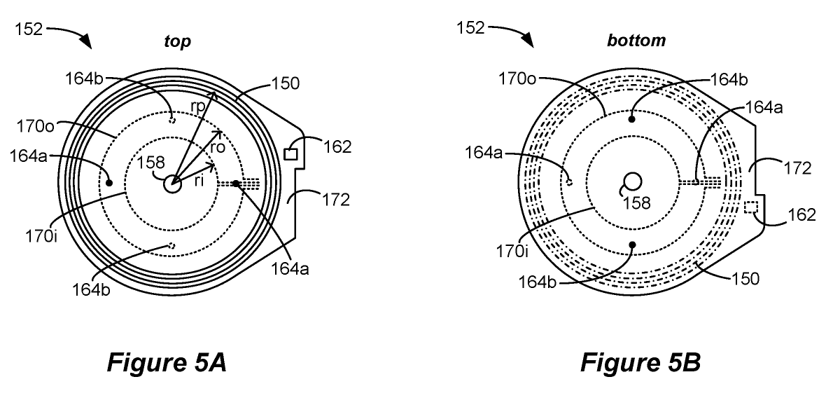

Various views of the circuit board 152 with housing portions 154a and 154b removed for easier viewing are shown in FIGS. 5A (top) and 5B (bottom). One or more bosses 156 (FIG. 4B) may be formed in the bottom housing portion 154b to fit within one or more holes 158 (FIGS. 5A and 5B) in the circuit board 152 to assist in affixing the circuit board in place within the housing 154. Top housing portion 154a may also have bosses or other stabilizing components, although this isn’t shown.

The circuit board 152 can be affixed in the housing 154 in other manners that are not shown, such as by snap fitting into clips formed into the bottom housing 154b. In another example, a thermally insulting material 160 (FIG. 4B) may be provided between the bottom of the circuit board 152 of the bottom housing 154b. This material 160 can comprise a foam tape having tacky surfaces on both of its sides to adhere to the bottom side of the circuit board 152 and to the inside surface of the bottom housing portion 154b. Preferably, this material 160 comprises a urethane foam, and more particularly a Poron™ urethane, manufactured by Rogers Corp. Use of a thermally insulating material 160 is preferred to prevent heat from the primary charging coil 150 from reaching the inside surface of the bottom housing portion 154b, which is most likely in contact with the patient during charging. Preferably, the thermal conductivity of thermally insulating material 160 is 0.2 W/m-K or less. In addition to providing thermal insultation, material 160 can help to dampen mechanical shock, thus preventing the charging coil assembly 102 from damage (e.g., if dropped). Material 160 is described in detail in U.S. Patent Application Publication 2018/0345025, which is incorporated herein by reference in its entirety.

Because the housing 154 of the charging coil assembly 102 is relatively thin—with a thickness x of 1.0 cm or less (FIG. 4B)—and because the primary charging coil 150 is generally located at the center of this thickness, the magnetic field 145 generated by the primary charging coil 150 during a charging session will generally be the same on both sides of the assembly. Therefore, and conveniently, either the top 154a or bottom 154b housing portion may face the patient (and the IMD 10) during a charging session.

Charging coil assembly 102 preferably includes at least one tuning capacitor 162 mounted to the circuit board 124. Capacitor 162 is coupled to the primary charging coil 150 (see FIG. 7) to tune the resonant frequency of this L-C circuit (e.g., to 80 kHz), and therefore to generally set the frequency of the AC magnetic field 145 (although this frequency is preferably adjustable, as explained further below). One skilled in the art will understand that the value of the capacitor 162 (C) connected to the primary charging coil 150 will be chosen depending on the inductance (L) of that coil and the desired frequency, in accordance with the equation f(res) = 1 / sqrt(2πLC). As one skilled in the art will appreciate, tuning capacitor 162 can be placed in series or in parallel with primary charging coil 126.

Also present in the charging coil assembly 102 are one or more temperature sensors 164, which are labeled 164a and 164b depending on whether such sensors are located on the top or bottom of the circuit board 152. As best shown in FIGS. 5A and 5B, two temperature sensors 136a are present on the top of circuit board 152, and two temperature sensors 136b are present on the bottom of circuit board, with each spaced 90-degrees within primary charging coil 150. In other examples, temperature sensors 164 may be present only on the top or only on the bottom of the circuit board 152. For example, only two temperature sensors 164a spaced at 180-degrees may be present on the top of the circuit board 152. The temperature sensors 164 may comprise thermistors, and in one example can comprise TMP112 High-Accuracy, Low-Power, Digital Temperature Sensors With SMBus and Two-Wire Serial Interface in SOT563, manufactured by Texas Instruments, Inc.

To assist with thermal management, and as explained in detail in the above-incorporated ‘025 Publication, the charging coil assembly 102 can further include a thermal diffuser 166 (FIG. 4B). In one example, the thermal diffuser 166 comprises a thermally conductive, soft plastic material, such as a non-silicone acrylic pad (e.g., Part No. 5590H, manufactured by 3M, Inc.), as discussed in the ‘025 Publication. Thermal diffuser 166 preferably has a tacky surface allowing it to be pressed onto and adhered to the top of the circuit board 152 and any components on this top surface, including temperatures sensors 164a. Thermal diffuser 166 preferably has a high thermal conductivity of greater than 1.0 W/m-K, and more preferably about 3.0 W/m-K. Thermal diffuser 166 also preferably has low electrical conductivity, and may have a dielectric constant of about 5-6 in one example. Thermal diffuser 166 preferably has a thickness greater than or equal to a thickness of the primary charging coil 150, which thickness may range from 0.5 to 4.0 mm for example. Because the thermal diffuser 166 is a soft material, it can be cut and shaped as necessary, as described in detail in the ‘025 Publication.

The thermal diffuser 166 is useful to dissipate heat away from the primary charging coil 150, which otherwise creates a hot ring-shaped area inside of the housing 154 when it is energized. Thermal diffuser 166 thus acts as a heat sink, and provides a heat transfer path away from the primary charging coil 150, thus distributing this heat over the diffuser’s larger circular area. Further, this distributed heat is directed to the temperature sensors 164a and/or 164b, particularly if in direct contact. As such, the temperature sensors are better able to accurately sense the temperature generated within the charging coil assembly 102.

To best distribute the primary charging coil 150’s heat, it is preferred that the thermal diffuser 166 be in contact with the primary charging coil, such as at an outer edge of the thermal diffuser, as shown in FIG. 4B. Although not shown, but as discussed in the ‘025 Publication, the thermal diffuser 166 may also overlap the primary charging coil 150. While it is beneficial to have the thermal diffuser 166 in contact with the primary charging coil 150, this is not strictly necessary. Heat may conduct from the primary charging coil 150 through the thermal diffuser 166 even if they are not in direct contact, as the heat may be transferred by intermediaries, such as the circuit board 152 or air within the charging coil assembly 102. In this regard, although not shown, the thermal diffuser 166 may be connected to one side of the circuit board 152 while the primary charging coil 150 is mounted to the other side.

As shown in FIGS. 5A and 5B, the charging coil assembly 102 further comprises inner and outer sense coils 170i and 170o. Generally speaking, these sense coils 170i and 170o are induced with a signal during generation of the magnetic field 145 from the primary charging coil 150. As discussed further below, the signals induced on the sense coils 170i and 170o are used for various purposes in the charger 100, such as to deduce alignment between the charging coil assembly 102 and the IMD 10, whether the power or frequency of the magnetic field 145 should be adjusted, and to receive back telemetry (e.g., LSK) from the IMD 10. These induced signals are further affected by the underlying IMD 10 being charged, which is also inductively coupled to the primary charging coil 150.

Although the sense coils 170i and 170o could comprise wire-wound coils similar to the primary charging coil 150, in the disclosed example they are formed as traces in the circuit board 152. More specifically, these coils 170i and 170o preferably comprise a single trace turn, although they could be formed with multiple turns in the circuit board’s traces as well. The sense coils 170i and 170o are preferably concentric with the primary charging coil 150, although this isn’t strictly necessary. Furthermore, and like the primary charging coil 150, the sense coils 170i and 170o are preferably circular, although they could have different shapes as well (e.g., squares, etc.). As their names imply, the outer sense coil 170o has a radius ro larger than the radius ri of the inner sense coil 170i. Furthermore, both of these radii are preferably smaller than the radius rp of the primary charging coil 150 as shown. However, this isn’t strictly necessary. In other examples, the radius of the outer sense coil ro could be larger than the radius of the primary charging coil rp, with the radius of the inner sense coil ri being smaller than rp. Still further, the radii of both the outer and inner sense coils ro and ri could be larger than rp.

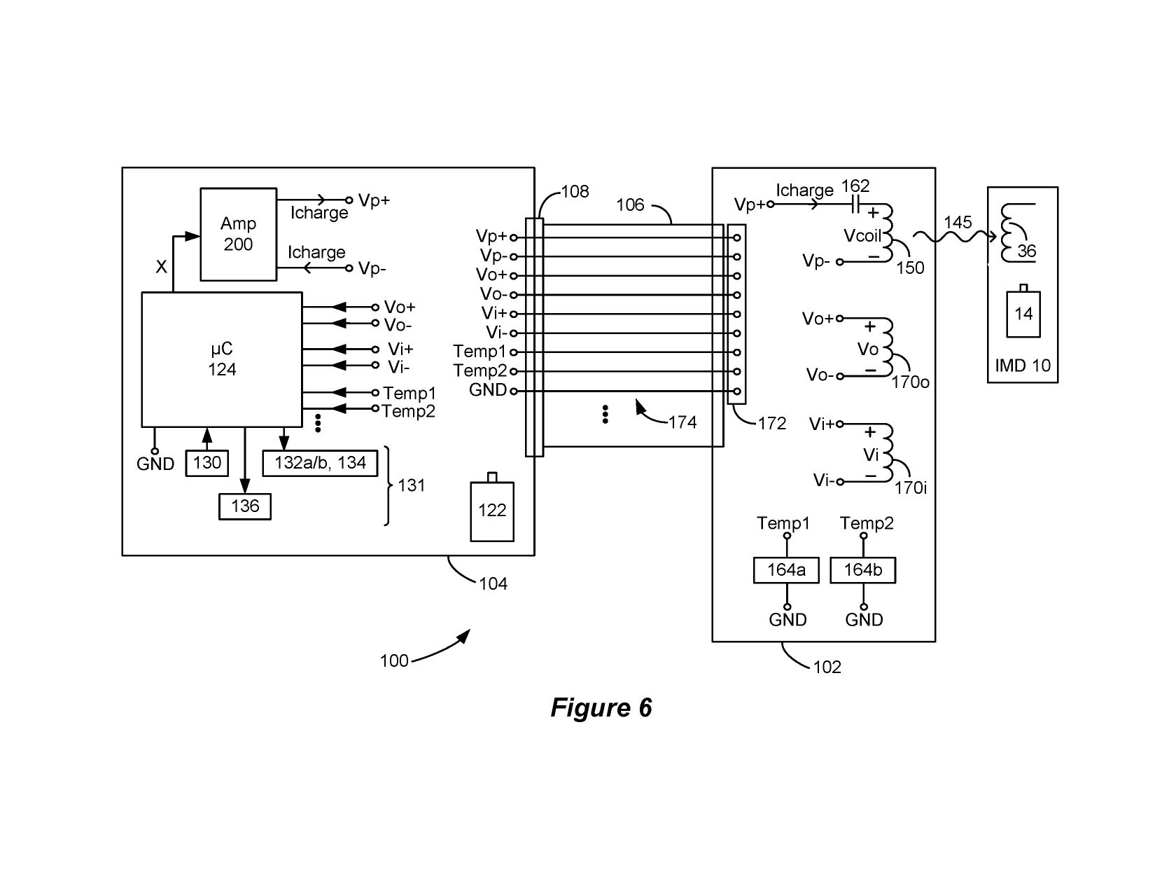

The circuit board 152 includes a contact portion 172 where wires 174 in cable 106 terminate and are connected to the components in the charging coil assembly 102, as best shown in FIGS. 4B and 6. One skilled in the art will understand that bond pads or similar contacts points would be present at the contact portion 172 to make these connections. In FIG. 6, connections are shown to both ends of the primary charging coil 150 (Vp+, Vp-); both ends of the outer sense coil 170o (Vo+, Vo-); both ends of the inner sense coil 170i (Vi+, Vi-); the temperature sensors 164 that are present (FIG. 6 assumes that two such sensors 164a and 164b are present, reporting two temperature Temp1 and Temp2); and a ground signal GND (as used e.g. by the temperature sensors 164). Although not shown, temperature data (Temp1 and Temp2) can be provided along a single I2C serial bus, and hence a single wire 174. Other wires 174 could also be included in the cable 106 as necessary to connect to other components in the charging coil assembly 102.

As shown in FIG. 6, signals from the sense coils 170i and 170o and the temperature sensors 164 are reported to the control circuitry 124 in the electronics module 104 and processed to useful ends, as discussed in more detail with respect to FIG. 7. As noted earlier, the charger 100 includes control circuitry 124 programmed via firmware with a number of functional modules whose functions are subsequently explained. These modules could also comprise discrete circuits, and thus need not necessarily comprise firmware programmed into control circuitry.

Temperatures reported by the sensors 164 (Temp1, Temp2) can be averaged by the control circuitry 124, and used to temporarily suspend the generation of the magnetic field 145 if the temperature gets too hot (above a maximum threshold). Once the temperature drops to a safe level (below a minimum threshold), the magnetic field 145 can again be started. As noted earlier, duty cycling the magnetic field 145 on and off this way based on temperature preferably occurs automatically and unbeknownst to the user.

As best shown in FIG. 7, the primary charging coil 150 is preferably driven using a class D amplifier 200, which is controlled by a digital drive signal X and its logical complement X*. This amplifier 200 has an H-bridge configuration comprising N-channel transistors 201a and 201b, and P-channel transistors 201c and 201d. When X is high (X* is low), transistors 201d and 201a are on and transistors 201b and 201c are off, thus causing current Icharge to flow through the primary charging coil 150 from constant power supply voltage Vcc to ground. When X is low (X* is high), transistors 201d and 201a are off and transistors 201b and 201c are on, thus causing current Icharge to flow through the primary charging coil 150 in the opposite direction. Therefore, Icharge is formed through the primary charging coil 150 as an AC current, which in turn produces magnetic field 145 as an AC field. For simplicity, drive signals X/X* are subsequently referred to singularly as drive signal X.

Drive signal X is output from pulse width modulator (PWM) 204, comprising firmware in the control circuitry 124. As shown, the drive signal X comprises a square wave with a particular on (a) / off (b) duty cycle. Programming on duration ‘a’ and off duration ‘b’ affects both the frequency f of the drive signal X (f = 1 / (a + b)) and the magnetic field 145. The frequency of the magnetic field 145 is nominally about 80 kHz, although this can vary in a range from 77 kHz to 87 kHz under control of a resonance module 216, as discussed further below. Frequency can also be temporarily set by an alignment module 210, again as discussed below.

Durations ‘a’ and ‘b’ also affect the duty cycle (DC = a / (a+b)) of the drive signal X, which adjusts the power of the magnetic field 145 under control of a power module 214, as discussed further below. For example, a higher duty cycle (up to 50%) will create a larger AC current Icharge, thus creating a larger-magnitude AC magnetic field 145, and in turn allowing the battery 14 in the IMD 10 to be charged faster. The duty cycle can also be temporarily set by the alignment module 210, as discussed below.

As noted above, the AC magnetic field 145 generated by the primary charging coil 150 will couple to and induce a signal in the outer and inner sense coils 170o and 170i, causing AC voltage Vo and Vi to build across each. Voltage Vo and Vi are also affected by coupling to the IMD 10. Voltages Vo and Vi are input to analog-to-digital (A/D) converters 203o and 203i to produce digital representations O and I of these waveforms. In the example shown, it is assumed that the control circuitry 124 includes (A/D) converters 203o and 203i at its inputs, but separate A/D circuitry could be provided as well if the control circuitry only includes digital inputs.

Signals O and I are continually determined when the charger 100 is producing the magnetic field 145. As explained further below, various other digital signals (Ao, Ai, θ, φ, and f) dependent on O and I and produced from other modules in the control circuitry 124 are likewise also continually determined. This allows the charger 100 to make adjustments continually during an IMD charging session, as explained further below. One skilled will understand that continually in this context means determining O and I, and dependent parameters Ao, Ai, θ, φ, and f periodically at a suitably high frequency, such as every tenth of a second or so.

AC signals Vo and Vi (and hence digitized signals O and I) will vary in accordance with the coupling of the magnetic field 145 between the charger 100 and the IMD 10. More specifically, this coupling will vary in accordance with the coupling between the primary charging coil 150 in the charging coil assembly 102 and conductive structures in the IMD 10, including the secondary charging coil 36 and the IMD 10’s conductive case. This coupling is affected by the alignment (in x and y directions) between the primary charging coil 150 and the IMD 10 as well and the distance z between them.

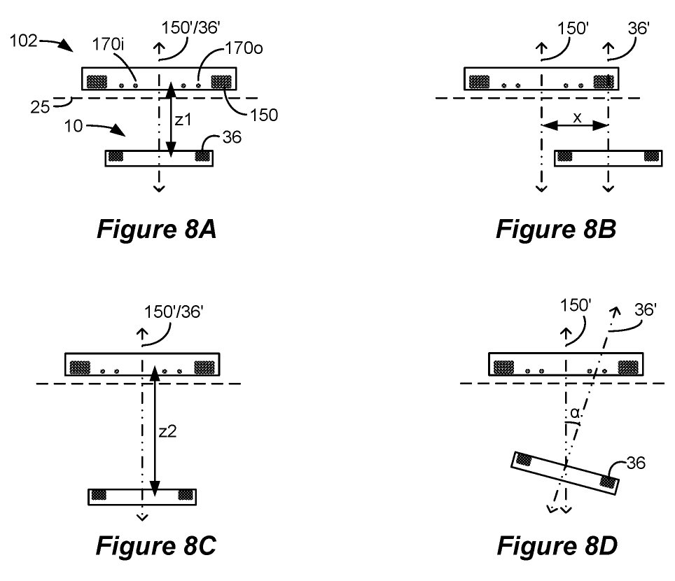

For example, the alignment is optimized between the primary charging coil 150 and the charging coil 36 in IMD 10 when the central axes of these coils 150’ and 36’ are collinear, meaning there is no x or y offset between the charging coils, as shown in FIG. 8A. By contrast, the alignment is poorer when these axes 150’ and 36’ are laterally shifted (in x and/or y), as shown in FIG. 8B. Poorer alignment implies poorer coupling, meaning that the magnetic field 145 isn’t as efficiently received by the IMD 14, lengthening the time it takes for the charger 100 to charge the battery 14. FIG. 8C shows the primary charging coil 150 and the IMD 10 to be well aligned, but the IMD 10 is implanted more deeply in the patient’s tissue 25. Compare z2 in FIG. 8C to z1 in FIG. 8A. Although aligned as best as possible, this also leads to poorer coupling. Coupling can also be negatively affected if the axes 150’ and 36’ are angled with respect to each other (α), which can occur for example if the IMD 10 is implanted at an angle in the patient (relative to the surface of the tissue 25), as shown in FIG. 8D. This is usually less significant however, as the IMD 10 as implanted and the charging coil assembly 102 are generally flat and relatively parallel.

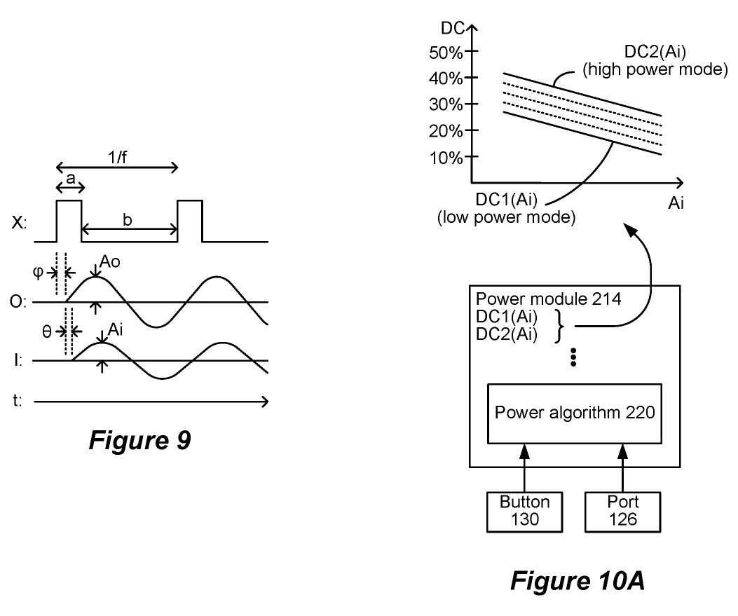

In any event, the coupling between the primary charging coil 150 and the IMD 10 affects both the amplitude and the phase of the AC signals induced on the sense coils 170o and 170i, as shown in FIG. 9. These induced signals as digitized (O, I) are shown in relation to the drive signal X. The signal O induced on the outer sense coil 170o has an amplitude Ao and a phase difference φ relative to the drive signal X. The signal I induced on the inner sense coil 170i has an amplitude Ai which is generally smaller than Ao because the inner sense coil 170i is farther from the primary charging coil 150 than is the outer sense coil 170o. Signal I, like O, has a phase difference relative to the drive signal X, and more specifically a phase difference θ relative to the outer induced signal O. In short, there is a phase difference φ between signal O and X, and a phase difference θ between signals I and O. Referring again to FIG. 7, phase difference φ is determined using signals O and X at a phase 1 module 208, while θ is determined using signals O and I at a phase 2 module 206.

As one skilled in the art will understand, phase differences such as θ and φ can comprise phase angles expressible in degrees or radians, or as time shift between the waveforms. For convenience, and preferably in an implementation, these phases differences are described herein as comprising phase angles. Nevertheless, phase differences can also be quantized as differences in time.

The charger 100 seeks to drive the frequency of the magnetic field 145 at the resonance frequency of the coupled charger/IMD system, and this occurs under control of the resonance module 216. This is preferred because driving the primary charging coil 150 at resonance will improve the efficiency of receipt of the magnetic field 145 at the IMD 10. This results in less power loss, and faster charging of the IMD’s battery 14. The resonant frequency can be expected to vary over time during a charging session, for example, if the charging coil assembly 102 moves (e.g., x, y, z, or angle α) relative to the IMD 10 due to patient movement. As such, the resonance module 216 preferably continually varies frequency f of drive signal X during a charging session to compensate for this variance to maximize power transfer efficiency, and this occurs as follows.

Phase angle φ between the drive signal X and the outer sense coil 170o (O) is reported to the resonance module 216, which will adjust (i.e., sweep) the frequency f until the phase angle equals zero (φ = 0). Alternatively, the frequency can be adjusted until the absolute value of the phase angle is smaller than a threshold (|φ| < φt), or is minimized. This establishes f at or near the current resonant frequency, thus maximizing the efficiency of power transfer to the IMD 10. The resonance module 216 can adjust the frequency in accordance with the polarity and magnitude of φ. For example, if φ is positive, f can be incrementally increased until φ = 0 is determined by the module 216. Frequency f can be incremented a significant amount if φ is significantly positive, and incremented a smaller amount if φ is only slightly positive. Likewise, if φ is negative, f can be similarly decremented by different amounts until φ = 0 is determined. Note that the resonance module 216 reports the current (adjusted) value of the frequency f to an alignment module 210, as discussed further below.

The resonance module 216 adjusts the frequency f by periodically programming the PWM 204 with appropriate on and off times (a and b) for the drive signal X. In so doing, the resonance module 216 preferably does not adjust the power of the magnetic field 145 being produced (which is instead preferably and independently controlled by the power module 214 described below). As noted earlier, the power of the magnetic field 145 can be adjusted by adjusting the duty cycle of the drive signal X (a / (a+b)). To keep this duty cycle constant when varying the frequency f (1 / (a + b)), the resonance module 216 will adjust both a and b proportionally. For example, when the resonance module 216 decreases the frequency, both a and b are increased from their current values by the same percentage, which keeps the duty cycle (and hence the power) constant.

Although the resonance module 216 is illustrated as directly programming on and off times (a and b) for the PWM 204, one skilled will understand that the PWM 204 can be programmed with information indicative of these on and off times, which may include the on/off times directly, or other information from which these on/off times can be gleaned. For example, the resonance module 216 may program the PWM with on duration ‘a’ and time period T (= 1/f = a+b), which information effectively provides on/off durations for the drive signal X that the PWM 204 will produce.

Although the resonance module 216 preferably considers and seeks to minimize phase angle φ between the drive signal X that drives the primary charging coil 150 and the outer sense coil 170o (O), the resonance module could consider and seek to minimize other phase angles as well, such as the phase angle between the drive signal X and the inner sense coil 170i (I). However, it is preferred to use phase angle φ because the outer sense coil 170o is closer to the primary charging coil 150. As such, the outer sense coil 170o (as compared to the inner sense coil 170i) is more strongly coupled to the primary charging coil 150, and, being larger in area, captures more the flux of the magnetic field 145. As such, phase angle φ comprises a convenient proxy to assess the primary charging coil 150, which is useful when adjusting frequency to resonance in the resonance module 216. Furthermore, the outer sense coil 170o is less affected by back EMF from the IMD 10 (i.e., an opposing magnetic field that is generated in the IMD 10 in response to the magnetic field 145). This back EMF, which can be important to detect for other reasons (as discussed further below), is not as important when adjusting the frequency.

Although the resonance module 216 is described as using a phase angle to adjust frequency to resonance, other means can be used as well, which do not necessarily involve the use of sense coils or phase angles. In another example, the primary coil 150 can be assessed directly, for example, by adjusting the frequency until the current through this coil is maximized.

As noted above, the power of the produced magnetic field 145 is preferably controlled via the power module 214, which also periodically programs the PWM 204 with appropriate on and off times (a and b) for the drive signal X (or information indicative of those times as explained earlier). Like adjustments to the frequency, it may be beneficial to adjust the power to compensate for the position and alignment of the charging coil assembly 102 relative to the IMD 10. For example, if the charging coil assembly 102 is misaligned or relatively far from the IMD 10 (either temporarily during the charging session due to patient movement, or simply because the IMD 10 is deeply implanted), this will reduce the coupling to the IMD 10 (see FIG. 8C), and thus reduce the power the IMD receives from the magnetic field 145. In this circumstance, it may preferable to increase the power of the magnetic field 145 to compensate to keep power reception at the IMD 10 relatively constant. Conversely, if the charging coil assembly 102 is closer and/or well aligned to the IMD 10, the power of the magnetic field 145 can be reduced to prevent over-charging or overheating.

As shown in FIG. 7, power adjustment at the power module 214 preferably occurs by considering the amplitude Ai of the signal induced on the inner sense coil 170i (see FIG. 9). Using the amplitude Ai of the inner sense coil 170i is preferred because (as compared to the outer sense coil 170o) it is not as strongly coupled to the primary charging coil 150, and is more strongly affected by back EMF from the IMD 10, and thus Ai operates as a better indicator of the IMD 10’s distance from the primary charging coil 150. This amplitude Ai is determined from digitized waveform I at an inner amplitude module 212. Ai preferably comprises a maximum amplitude for signal I as shown, but could also comprise any DC voltage indicative of I’s magnitude.

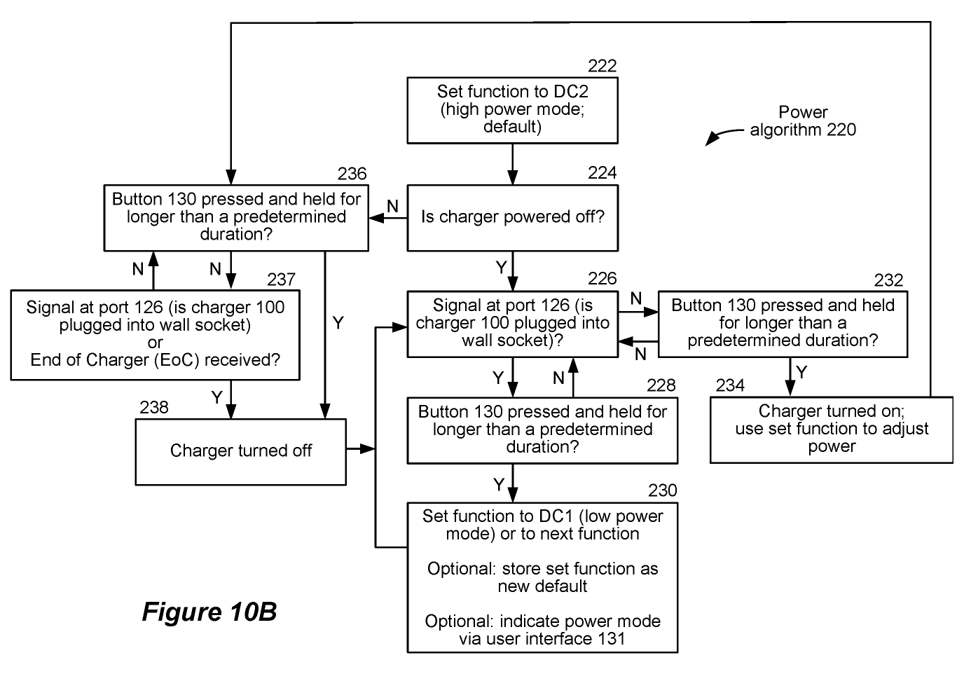

The power module 214 periodically receives this value Ai, and adjusts the power (sets the duty cycle of the drive signal X) in accordance with Ai via a function, DCx(Ai). FIG. 10A shows two such functions, DC1 and DC2, which are programmed into the power module As depicted, these functions linearly relate amplitude Ai and duty cycle DC values, but this is not strictly necessary, and functions DCx can be empirically determined based on testing.

Further, as shown, these functions inversely relate amplitude Ai and duty cycles DC values, with DC decreasing as Ai increases and vice versa. Again, this is not strictly necessary, but is preferred. This inverse relationship is preferred to correct for differences in the depth of the IMD 10. If the IMD 10 is relatively shallow (e.g., close to the charging coil assembly 102; FIG. 8A), Ai will decrease due to increased back EMF from the IMD 10. This increased reflected impedance will also affect the primary charging coil 150 coil. As a result, it is preferable to drive the primary charging coil 150 harder (with a higher DC) to compensate and to provide more power when Ai is low. By contrast, if the IMD 10 is relatively deep (e.g., farther from the charging coil assembly 102; FIG. 8C), Ai will be higher. This will decrease the reflected impedance, meaning the primary charging coil 150 need not be driven as hard to deliver suitable power, and thus the duty cycle can be decreased at higher Ai values.

Function DC1 sets lower duty cycles for the drive signal X and hence implements a low power mode of operation for the charger 100 (generation of a lower magnitude magnetic field 145), while function DC2 sets higher duty cycles for the drive signal X and hence implements a high power mode (generation of a larger magnitude magnetic field 145). Although only two function DCx are illustrated, more than two functions could be used in the power module 214 to provide even finer granularity of the powers that can be produced, as shown in dotted lines in FIG. 10A.

As noted, the functions DCx determine a duty cycle for the drive signal X in accordance with the current value of Ai being reported, and the power module 214 can program the PWM 204 with appropriate on and off times (a and b) for the drive signal X as necessary to affect that determined duty cycle (a / (a+b)). When adjusting the duty cycle (power), the power module 214 preferably does not adjust the frequency of the magnetic field 145 being produced (which is instead preferably and independently controlled by the resonance module 216, as explained earlier). For example, if the power module 216 needs to decrease the duty cycle to decrease the power of the magnetic field 145, it can decrease ‘a’ by an amount (a-Δ) but also increase ‘b’ (b+Δ) by the same amount. Likewise, if the power module 216 needs to increase the duty cycle to increase the power, it can increase ‘a’ by an amount (a+Δ) but also decrease ‘b’ by the same amount (b-Δ). Adjusting a and b this way adjusts the duty cycle while preserving the (resonant) frequency established by the resonance module 216 (1 / (a + b)).

Although the power module 214 preferably considers the amplitude on the inner sense coil 170i (Ai) for the reasons explained above, the amplitude of the outer sense coil 170o (Ao) could also be individually considered and used to adjust the power of the magnetic field 145.

Note that both the resonance module 216 and the power module 214 can periodically adjust the on and off times (a and b) for the drive signal X during a charging session. To preserve the independence of these two modules, and to prevent conflicts, it is preferred that the resonance module 216 and the power module 214 not adjust these parameters at the same time. Instead, it is preferred to interleave the adjustments made by these modules 216 and 214 in time.

As discussed above, the particular function DCx implemented by the power module 214 to set the relative power of the magnetic field 145 is selectable to allow the charger 100 to operate in low and high power modes (and other intermediate modes if more than two functions are used). This is beneficial because charging an IMD 10 will necessarily cause the charger 100 to heat somewhat. If a user finds the charger 100 to be uncomfortably hot, the user can select use of a lower power mode (e.g., DC1). This causes the charger 100 to produce the magnetic field 145 with less power, which will reduce the charger’s temperature, but will also charge the IMD 10’s battery 14 more slowly. If a user desires to charge the IMD 10 more quickly, and can tolerate higher temperatures, the user can select use of a higher power mode (e.g., DC2). As discussed further below, the user can set or toggle between power modes using button 130 on the user interface 131 of the electronics module 104.

Selection of a power mode, and operation of the button 130 more generally to turn the charger on and off, can be implemented by use of a power algorithm 220, as shown in FIG. 10B. As shown, this algorithm 220 can be programmed as part of the power module 214, but could also reside elsewhere in the IMD’s control circuitry 124.

As a first step 222, the algorithm 220 will set a particular default function for use in adjusting power. In this example, that function is DC2, which implements the high power mode for faster IMD charging. However, another function and power mode can be selected later, and possibly stored as the default for use in subsequent charging sessions, as discussed below.

At step 224, the algorithm 220 queries whether the charger 100 is currently powered off, or is on and in the process of generating a magnetic field 145 to charge the IMD 10. Note that if the charger 100 is currently generating a magnetic field, some function DCx (presumably the default function DC2 as set at step 222) is being used by the power module 214 to adjust its power and to set use of a particular (high) power mode. This function and power mode can be changed as discussed further below. In any event, if the charger 100 is currently on, step 236 assesses whether the button 130 has been pressed, indicating that the user wishes to turn off the charger. To ensure that the button 130 has not been inadvertently and only transiently pressed, step 236 may more specifically determine whether the button 130 has been pressed and held as depressed for longer than a predetermined duration, such as one second. If so, the charger is turned off at step 238, thus stopping generation of the magnetic field 145.

Other assessments can also be made by the power algorithm 220 to turn off the charger 100. For example, and as shown in step 237, the charger can be turned off if a signal is detected at the charger’s port 126 (FIG. 4A and 4B). The presence of the signal at port 126 would normally indicate that the charger 100 has been plugged into a wall socket or another power source, such as when the charger’s battery 122 needs to be recharged, or if the charger 100 needs to receive new operating software. Providing a signal at the port 126 can be used for other purposes in the power algorithm 220, as discussed later. The power algorithm 220 can also at step 237 turn off the charger 100 if an End of Charge (EoC) signal has been received at the charger 100, as discussed elsewhere in this disclosure. Once the charger 100 has been turned off at step 238, the algorithm can proceed to step 226, as discussed next.

Returning to step 224, if the charger 100 is currently powered off (or was turned off at step 238), the power algorithm 220 can assess whether a signal is present at the port 126 at step 226. If not, the algorithm 220 at step 232 assesses whether the button 130 has been pressed, indicating that the user wishes to turn on the charger. Again, to ensure that the button 130 has not been inadvertently pressed, step 232 may more specifically determine whether the button 130 has been pressed and held for longer than a predetermined duration, such as one second. If so, the charger is turned on at step 234 to start generating the magnetic field 145. Because the function to be used by the power module 214 was set earlier (e.g., DC2; step 222) and has not been changed, this function will be used to adjust the power of the magnetic field 145, which in this example implements use of the high power mode.

By contrast, if a signal is present at the port 126 at step 226, pressing of the button 130 is used to set use of a different function, and hence to set a new power mode. In this circumstance, if the button 130 is pressed (and again optionally held for a predetermined duration) at step 228, a different function is set (e.g., DC1) at step 230, which in this example implements use of the low power mode. (This predetermined duration which the button 130 is held at step 228 can be the same duration that is assessed when determining whether the charger should be turned on (232) or off (236). Alternatively, this duration could be set to a different (e.g., longer) duration at step 228 (such as two seconds)).

The new function and power mode set at step 230 will be used the next time the charger 100 is turned on. Accordingly, and reverting back to step 226, when the user later removes the signal at the port 126 (e.g., unplugs the charger from the wall socket), and presses and holds the power button (232), the charger is turned on and will use the new function (DC1) set earlier at step 230 (234).

Other optional actions can be taken at step 230 as well. For example, when a new function is selected (e.g., DC1), that function can be stored by the charger 100, and used as the new default (at step 222) for subsequent charging sessions. Alternatively, and perhaps after some period of time, the power algorithm 220 can revert to use of the default function and power mdoe programmed by the manufacture, even if a new function was (temporarily) set at step 230 for use during a particular charging session. This alternative might require the patient to continually set the function and power mode to his desired power mode during each subsequent charging session. While this is less preferred, it still has utility, particularly if use of manufacturer’s programmed default power mode is generally warranted for safety or other reasons.

Although power algorithm 220 has thus far been described assuming the use of only two functions DC1 and DC2 (implanting low and high power modes), with step 230 being used to toggle between these, step 230 can alternatively be used to set use of a next power mode (e.g., DCx) if more than two are present. Thus, by pressing and holding button 130 a number of times to loop through steps 226-230, the user can scroll through and set use of the various functions (e.g., DC1, then DC2, then DC3, then DC4, then back to DC1, etc.) to implement use of a wider variety of power modes.

Another optional action that can be taken at step 230 is to indicate the selected power mode via the charger’s user interface 131. As an example, selection of the low power mode (DC1) may be indicated by illuminating the implant battery LED 132b (FIGS. 4A and 4B) red, while selection of the high power mode (DC2) may be indicated by illuminating the implant battery LED 132b (FIGS. 4A and 4B) green. The selected power mode (the set function) can alternatively or additionally be indicated using the speaker 136, with selection of the low power mode (DC1) indicated by one beep, and selection of the high power mode (DC2) indicated by two beeps. Still other indications could be provided via the user interface 131 reflective of the selected power mode or function.

The following summarizes a typical scenario in which the power algorithm 220 as described may operate. With the charger 100 not plugged in at port 126 (e.g., to a wall socket) and currently off, a patient presses (and holds) the button 130 (step 232) to turn on the charger 100 to charge their IMD 10 (234). By default, this comprises use of the default function DC2 to adjust the power (222), which implements use of the high power mode and the generation of a higher-power magnetic field 145 (234). If the patient finds the temperature to be uncomfortably hot, the user can then press (hold) button 130 again (step 236) to turn the charger off (238). The patient can then plug the charger 100 into a wall socket at port 126 (step 226), and then press (hold) the button 130 to set a (next) function DC1 to implement use of a low power mode (230). The user can then unplug the charger 100 (step 226), and again press (hold) the button 130 (232), which will again turn on the charger 100, using the newly-set function DC1 to adjust the power and implement use of the low power mode and the generation of a lower-power magnetic field 145 (234).

If the patient finds use of this low-power mode to take too long to charge the IMD 10, this process can be repeated and a new (higher) power mode selected: the patient can press the button 130 to turn off the charger; plug in the charger; press (hold) the button 130to select use of a (next) higher power mode (e.g., DC2); unplug the charger; and then press (hold) the button 130 to use this newly selected power mode.

It should be understood the selection of a new power mode via use of power algorithm 220 does not necessarily require use of a particular function DCx (that relates amplitudes Ai to a duty cycle DC). Instead, power algorithm 220 can be used to select use of different powers for the magnetic charging field 145 without relation to functions as described.

As described, the power algorithm 220 preferably requires that the charger be plugged in (226), and that the button 130 be pressed and held (228), before the power mode (function) is changed (230). These steps are preferred as a safeguard against inadvertent switching of the power mode, but are not strictly required when selecting different power modes.

For example, adjustment of the power mode can be made by the power algorithm 220 without considering whether the charger is plugged in at port 126, and instead can be made based on the duration of the button 130 press. Thus, in an alternative, the algorithm 220 can simply at steps 226 and 228 assess if the button 130 has been pressed for a sufficient duration above a threshold, and if so will change the power mode at step 230, without assessing whether the charger is plugged in at the port 126. By contrast, if the duration of the button 130 press is below the threshold, the charger 100 can instead be turned on (234) or off (238).

In another alternative, the power algorithm 220 can simply assess if the button 130 is pressed while the charger is plugged in at the port 126 without additionally assessing the duration that the button 130 is pressed. That is, step 228 may only assess if the button 130 is pressed, but may not consider whether it is held as pressed (for some particular duration).

Signals other than those at the port 126 can also be used in the power algorithm 220 and queried at step 226 to provide a safeguard against inadvertent switching of the power mode. For example, the charger 100 could include an additional button (not shown) or other user interface element that must be selected at step 226 if it is desired to switch power modes. In another example, step 226 may consider a signal from one or more of the temperature sensors 164. These sensors 164 would register a higher temperature if the charging coil assembly 102 is in contact with the patient. Thus step 226 may query this temperature signal to see if it is high (relative to a threshold), and only then allow the power mode to be switched (230) when the button 130 is pressed (228). In short, step 226 can be modified to consider signals other than those present at the port 126.

Button 130 should be understood as comprising any user interface element that is selectable by a user to set the power mode of the charger 100, and also possibly to turn on and off the charger. Such user interface elements can comprise a button as disclosed, but could also comprise a switch, a touch-sensitive element on a display, etc.

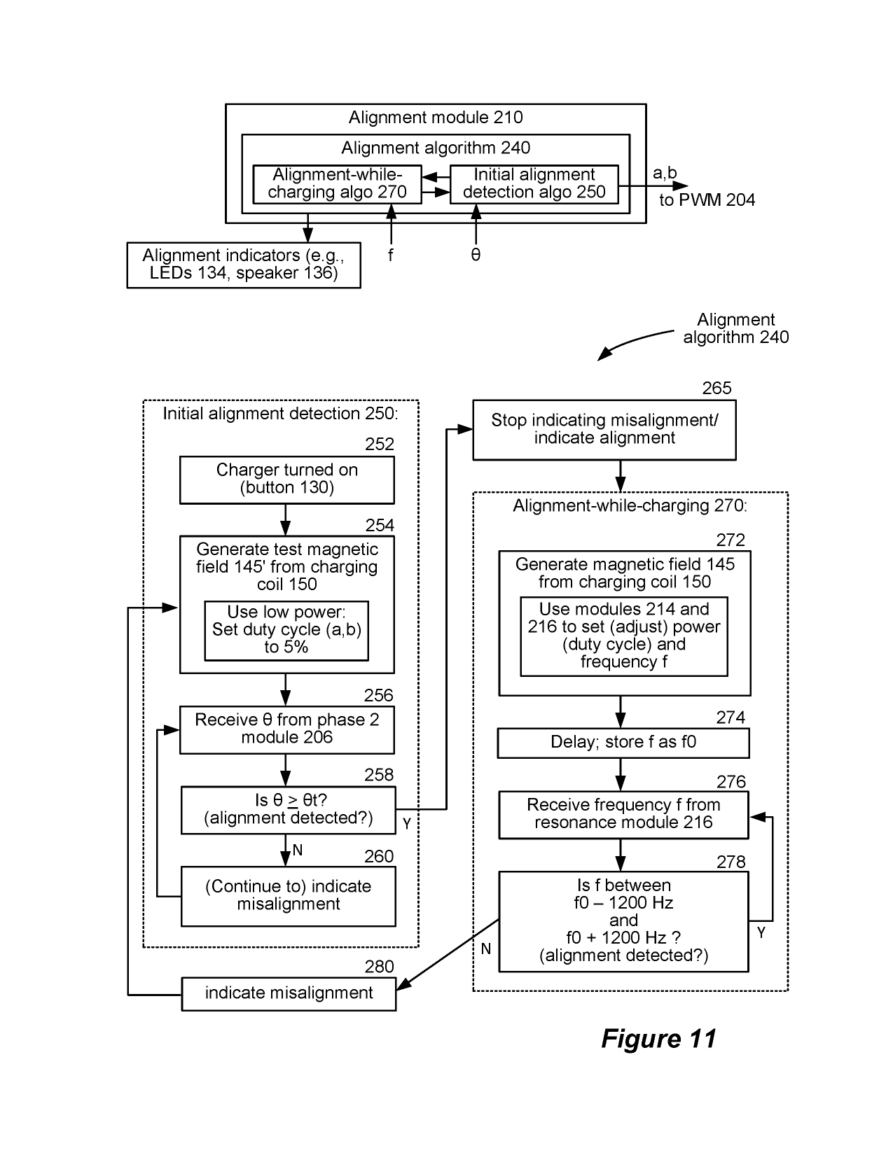

Alignment between the primary charging coil 150 and the IMD 10 is determined in an alignment module 210, which is explained in more detail with reference to FIG. 11. As shown, the alignment module 210 can implement an alignment algorithm 240. This algorithm 240 preferably comprises algorithms 250 and 270, which are briefly explained before a more thorough description of each.

The first algorithm 250 is referred to as an initial alignment detection algorithm, and determines whether the charging coil assembly 102 is in proximity and well aligned to the IMD 10 so that charging can begin in earnest (via generation of a magnetic charging field 145). The initial alignment detection algorithm 240 preferably occurs at the beginning of a charging session (when the charger 100 is turned on via button 130), but can also occur iteratively during a charging session, as explained further below. Generally speaking, initial alignment detection algorithm 250 comprises a form of alignment detection between the charging coil assembly 102 and the IMD 10, and when making this detection uses the phase angle θ (from phase 2 module 206) between the signals O and I induced on the outer and inner sense coils 170o and 170i.

If the initial alignment detection algorithm 250 determines that the charging coil assembly 102 is proximate to the IMD 10, use of the second algorithm 270 can begin. This second algorithm 270 is referred to as an alignment-while-charging algorithm 270, and as its name implies allows charging to begin in earnest (via magnetic field 145). Algorithm 270 also determines whether the alignment between the charging coil assembly 102 and the IMD 10 is proper, and when making this determination uses the current value of the frequency f, which as noted earlier may be adjusted by the resonance module 216. If the alignment is deemed improper by algorithm 270, the alignment algorithm 240 can revert to use of the initial alignment detection algorithm 250.

Although the initial alignment detection algorithm 250 and the alignment-while-charging algorithm 270 are described as part of the alignment algorithm 240, it should be understood that each of algorithms 250 and 270 can be used individually. That is, the initial alignment detection algorithm 250 can be used by itself to determine the charging coil assembly 102’s proximity to or alignment with the IMD 10, while the alignment-while-charging algorithm 270 can be used to determine alignment between the charging coil assembly 102 and the IMD 10 while charging.

The initial alignment detection algorithm 250 occurs first, and is initiated when the charger 100 is turned on (using button 130) at step 252. At step 254, the algorithm 250 causes the charger 100 to produce a test magnetic field 145’. Preferably, this test field 145’ is of a relatively low power, i.e., lower than the power that would typically be present in the magnetic field 145 when charging the IMD 10 (as occurs later during algorithm 270). The test field 145’ may charge the IMD 10 somewhat, but probably not significantly due to its low power.

Because power can be set by adjusting the duty cycle (a / (a+b)) of the drive signal X as discussed earlier, this step 254 sets a low duty cycle (e.g., 5%) by setting values ‘a’ and ‘b’ accordingly at the PWM 204. These parameters also set the frequency f (1 / (a+b)), which is set to a nominal value (e.g., 80 kHz). In short, the duty cycle (power) and frequency of the test field 145’ are preferably constant, and the power module 214 and resonance modules 216 are preferably disabled during algorithm 250 and thus will not adjust the power or frequency. While it is preferred the test field 145’ be of relatively low power, this is not strictly required, and instead the field 145’ can also generally equal the power of the magnetic field 145 that will be generated later during algorithm 270.

Producing the test field 145’ induces signals on the outer and inner sense coils 170o and 170i, which as noted earlier are digitized (O and I) and sent to phase 2 module 206 to determine a phase angle θ between them. This phase angle θ is periodically received by algorithm 250 at step 256, and is indicative of the charging coil assembly 102’s proximity to the IMD 10: if the assembly 102 is nowhere near the IMD 10 (and thus not producing a back EMF), waveforms O and I will be in phase, and thus the phase angle θ will equal (or be near) zero. As the charging coil assembly 102 approaches the IMD 10, this phase angle θ will start to increase. Therefore, at step 258, algorithm 250 compares the phase angle θ to a threshold θt (e.g., 2 degrees) that is indicative of good coupling between the charging coil assembly 102 and the IMD 10, and hence indicative of the assembly’s proximity to the IMD. If phase angle θ is below θt at step 258, the initial alignment detection algorithm 250 determines that the charging coil assembly 102 is not (yet) proximate to the IMD 10. This would often be the case if the patient is initially trying to position the charging coil assembly 102 on their person relative to the IMD 10 or is placing the charging coil assembly 102 into their charging belt, etc.

Assessment of the phase angle θ between the outer and inner sense coils 170o and 170i is preferred during the initial alignment algorithm 250, because loading due to the implant (back EMF) will tend to affect the phase of the inner sense coil 170i more than the outer sense coil 170o, causing a large phase shift. That being said, other phase angles, such as the phase angle between the outer sense coil (φ) or the inner sense coil and the drive signal X could be used as well.

If alignment has not been detected at step 258, the algorithm 250 preferably indicates misalignment via the user interface 131 at step 260. As noted earlier, this can involve setting alignment LEDs 134 (yellow), and/or issuing beeps from the speaker 136. Indicating misalignment to the patient is helpful, as this notifies them that the positioning of the charging coil assembly 102 relative to the IMD 10 needs physical adjustment. Thereafter, the algorithm 250 reverts to step 256 where a new value for phase angle θ is considered. If alignment continues to not be detected (i.e., θ is still below θt), steps 256-260 can loop to continue to monitor θ.

As the patient continues to position the charging coil assembly 102, it would be expected that phase angle θ would eventually rise above the threshold θt set at step 258 (e.g., θ > θt). This indicates that the charging coil assembly 102 is now proximate the IMD 10. At this point, any misalignment indicators set earlier (260) can be extinguished, and/or such indicators can be changed to indicate alignment, at step 265. Thus, LEDs 134 can now be set green (or simply turned off), and speaker 136 may stop emitting beeps.

Preferably, the phase angle threshold θt is set to a value that ensures particularly good coupling between the charging coil assembly 102 and the IMD 10. In this way, the initial alignment detection algorithm 250 further ensures that the charging coil assembly 102 is well aligned and in a good position to begin charging the IMD 10, as occurs in next steps. A suitable value for θt can be determined empirically through experimentation.

With the charging coil assembly 102 now in proximity and good alignment to the IMD 10, alignment algorithm 240 can proceed to use of the alignment-while-charging algorithm 270. During this algorithm 270, charging of the IMD 10 can be begin in earnest via generation of a magnetic charging field 145 at step 272. At this point, the power module 214 and the resonance module 216 can be enabled to operate as described above to respectively adjust the power (in accordance with Ai and the power function DCx selected) and the frequency f (using phase angle φ) of the magnetic field 145.

At step 274, a short delay can be implemented to allow the power module 214 and the resonance module 216 time to operate and stabilize the power and frequency f of the magnetic field 145. After this delay, the current value for the frequency f is stored as f0. f0 will vary from patient to patient, and even between different charging sessions for the same patient, depending on the coupling (positioning) between the charging coil assembly 102 and the IMD 10. In any event, initial frequency f0 comprises a frequency f that is indicative of good coupling and alignment between the charging coil assembly 102 and the IMD 10, particularly because good coupling and alignment has just recently been established (via θt) during the initial alignment detection algorithm 250.

After determining f0, and while the charger 10 is charging the IMD 10, the alignment algorithm 240 continues to periodically monitor the frequency f issued from the resonance module 216 at step 276. Monitoring frequency f is useful because, as explained above, this frequency is continually being adjusted by the resonance module 216 as the coupling between the charging coil assembly 102 and the IMD 10 changes during the charging session. Such coupling changes can readily occur if the charging coil assembly 102 is not well secured and thus changes position relative to the IMD 10, as may occur if the patient moves for example.

As such, the frequency f may over time during the charging session begin to shift from its initial and stored value of f0. Such shifts in f away from f0 suggest that the coupling and hence alignment is worsening. Small shifts in f can be acceptable and may not significantly impact IMD 10 charging, but if frequency f shifts from f0 too much, the IMD 10 will not as efficiently receive power from the magnetic field 145, which can slow the charging of the IMD’s battery 145.

Therefore, at step 278, a range or guardband of frequency values is used to determine whether f has shifted too far from f0, and hence whether the charging coil assembly 102 has become misaligned relative to the IMD 10. In the example shown, this guard band comprises a +/- 1200 Hz range around f0. In other words, if f0 – 1200 < f < f0 + 1200, step 278 determines that the charging coil assembly 102 is still reasonably (if not perfectly) aligned with the IMD 10. Therefore, charging and production of the magnetic field 145 can continue as normal, and the alignment-while charging algorithm 270 can return to step 276 to periodically receive updated values of the frequency f from the resonance module 216.

Eventually, the frequency f may shift outside of the guardband (i.e., f < f0 – 1200 or f > f0 + 1200). In this case, step 278 determines that the charging coil assembly 102 has now become misaligned to the IMD 10. At this point, the alignment algorithm 240 can indicate misalignment to the patient via the user interface 131 (step 280), similar to what was described earlier at step 260 (alignment LEDs 134 lit yellow, issuing beeps from the speaker 136, etc.).

Further, the algorithm 240 can return to use of the initial alignment detection algorithm 250. As such, the charger 100 may discontinue generating magnetic field 145 and instead again begin generating a low-power test field 145’ (step 254) as described earlier. By way of review, the test field 145’ is produced using a constant power and frequency, and without use of the power and resonance modules 214 and 216. Once again, phase angle θ can be considered and compared to threshold θt (steps 245, 258), and misalignment can (continue to) be indicated at step 260, which informs the patient to adjust the positioning of the charging coil assembly 102 relative to the IMD 10.

Once this positioning is fixed and good coupling and alignment are again established (θ > θt), misalignment indications can cease (step 265), the alignment-while-charging algorithm 270 can be re-entered, and generation of magnetic field 145 can begin again (step 272). Because the charging coil assembly 102 may be differently positioned, a different frequency f is preferably established (as determined by the resonance module 216) and stored as a new (updated) value for f0 (274).

To summarize, the alignment algorithm 240 allows the initial alignment detection algorithm 250 and the alignment-while-charging algorithm 270 to be used iteratively during a charging session. The initial alignment detection algorithm 250 preferably establishes good alignment between the charging coil assembly 102 and the IMD 10, and thereafter algorithm 240 allows charging to begin in earnest during the alignment-while-charging algorithm 270. Should the alignment thereafter become poor, algorithm 240 reverts again to use of the initial alignment detection algorithm 250 to once again establish good alignment before charging in earnest can continue, etc.

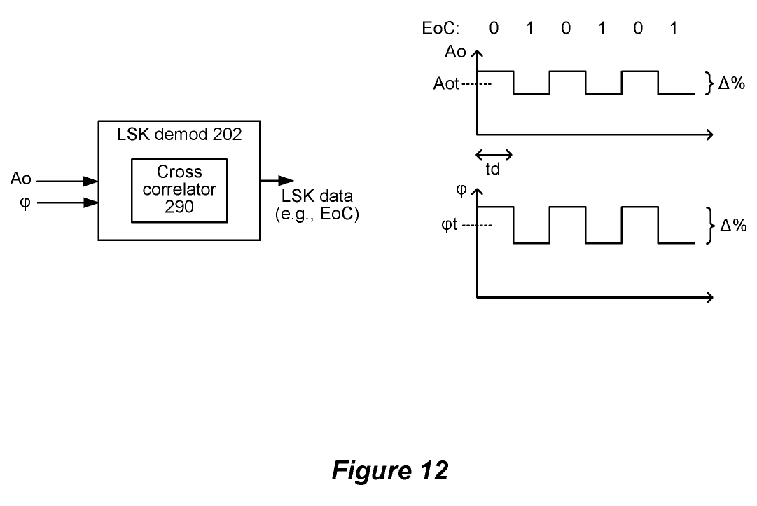

Receipt of Load Shift Keying (LSK) telemetry from the IMD 10 is handled in the charger 100 by a LSK demodulator 202 (FIG. 7), which is described in more detail with respect to FIG. 12. As described earlier, an IMD 10 being charged can telemeter an end-of charging (EoC) signal (or other LSK data) to a charger (50, 100) to indicate that the charger can suspend production of its magnetic field. This EoC signal can be transmitted by the IMD 10 via operation of an LSK modulator 74 in the IMD 10, which operates to either short (LSK = 1) or unshort (LSK = 0) the IMD 10’s secondary coil via switches 74. This EoC signal may comprise an alternating sequence of some number (e.g., 128) of bits (e.g., 010101…), each having a bit duration td.

As explained earlier, LSK telemetry causes reflected impedances as the magnetic field 145 is being produced, and this reflected impedance can be assessed at the LSK demodulator 202 to recover the EoC signal (or other LSK data). LSK-based reflections can sensed in a number of ways in the charger 100 that preferably involve use of one or more of the sense coils 170o and 170i.

In one example, the amplitude Ao of the outer sense coil 170o is assessed at the LSK demodulator 202. This outer amplitude Ao is determined at an outer amplitude module 218 (FIG. 7) from digitized signal O. Amplitude Ao preferably comprises a maximum amplitude for signal O (FIG. 9), but could also comprise any DC voltage indicative of O’s magnitude. As explained earlier, reflections caused by the LSK data (e.g., EoC) will cause the voltage across the primary charging coil 150 (Vcoil, FIG. 7) to change depending on the logic state (0 or 1) of the LSK data being transmitted by the IMD 10.

The LSK data also affects the voltage Vo across the outer sense coil 170o, which is coupled to both the primary charging coil 150 and the IMD 10. Thus, Ao also changes in accordance with the LSK data, as shown in FIG. 12. Sensing these reflections at the outer sense coil 170o is preferred to sensing them at the primary charging coil 150 (compare FIG. 3), because the primary charging coil 150 need not be connected to (and isn’t loaded by) the LSK demodulation circuitry 202. This makes it easier to tune and adjust the driving (X) of the primary charging coil 150 (via modules 214 and 216 as described earlier). It is preferred to sense the amplitude of the outer sense coil Ao (as opposed to the amplitude of the inner sense coil Ai) when sensing LSK telemetry because Ao provides a more sensitive indication of this telemetry when the IMD 10 is implanted relatively deeply. That being said, the amplitude of the inner sense coil Ai could also be assessed, which may be preferably when the IMD 10 is implanted more shallow.

In another example, the phase angle φ between the drive signal X and the signal induced on the outer sense coil 170o is assessed at the LSK demodulator 202. As described earlier, this phase angle φ is determined at a phase 1 module 208 (FIG. 7), and as shown in FIG. 12 this angle φ is also affected by the logic state (0 or 1) of the LSK data (e.g., EoC) being transmitted by the IMD 10. While it is preferred that the LSK demodulator 202 assesses the phase angle φ between the drive signal X and the signal induced on the outer sense coil 170o, other phase angles involving the sense coils could be used as well. For example, the LSK demodulator 202 could also assess the phase angle between the inner sense coil 170i and the drive signal X, or the phase angle between the outer and inner sense coils 170o and 170i (i.e., θ, as determined by phase 2 module 206). Note that signals Ao and φ in FIG. 12 are shown ideally, and in reality may be noisier.

In either case, the LSK demodulator 202 assesses the received signals (e.g., Ao or φ) to determine the digital logic states of the transmitted LSK data. This involves assessing both the timing at which changes occur as well as the magnitude of the changes. For example, the LSK demodulator 202 can be programmed with the bit duration (td) at which LSK data is transmitted from the IMD 10, and hence assess whether the magnitude of Ao or φ is transitioning in accordance with that timing. The LSK demodulator 202 can also assess the magnitude of these signals at those transitions. For example, the demodulator 202 can compare the magnitudes to thresholds (Aot or φt) to determine whether a logic 0 or 1 has been received. Because the magnitudes of Ao or φ can change as the charger 100 operates and as coupling the with IMD 10 changes, it is preferred that these thresholds would change or scale as well. In another example, the LSK demodulator 202 may instead determine the logic state of the received signal by assessing a percentage change (Δ%) in the received magnitudes.

In a preferred example, more than one signal is assessed by the LSK demodulator 202 to recover the telemetered LSK data (e.g., EoC), and it is particularly preferred to receive and assess both Ao and φ. When assessing more than one signal, a cross correlator 290 can be used. This cross correlator 290 can operate on the received signals before or after they assigned particular digital logic values. Cross correlating the two received signals is preferred to improve the reliability of the received LSK data. For example, when considering the EoC signal (010101…), one or more of the transmitted bits as represented by Ao may not be reliably received (e.g., 011101…). Likewise, the transmitted bits as represented by φ may not reliably received (e.g., 010100…). Cross correlating the time-varying values of Ao and φ allows the LSK modulator 202 to determine that the EoC signal (or other LSK-telemetered data) has been received, even though that data may not be perfectly indicated by assessing each of the signals individually. While it is preferred to sense (and cross correlate) both Ao and φ, any two other signals indicative of the transmitted LSK data can be assessed as well (e.g., Ao and θ, Ao and Ai, Ai and any phase angle, etc.).

Regardless of the number of signals assessed, the LSK demodulator 202 will recover the transmitted LSK data and inform the charger’s control circuitry 124 accordingly. Thus, if a pattern corresponding to the EoC signal is received, the control circuitry 124 will suspend charging, and may provide corresponding indications via the charger’s user interface 131 (e.g., lighting LED 132b green (FIG. 4B); issuing a continuous double beep for a duration, etc.). Such control may involve suspending the issuance of drive signal X at the PWM 204, or more simply may turn off the charger 100.

In other examples where the recovered LSK data indicates charging parameters telemetered from the IMD 10 that may be useful in controlling charging (e.g., the IMD’s charging current Ibat, the voltage of the IMD’s battery 14 Vbat, or a temperature in the IMD), the LSK demodulator 202 can provide such data to appropriate modules in the charger 100. For example, recovered data may be provided to the power module 214 to adjust the power of the magnetic field. If the IMD 10 being charged has the capability to determine whether it requires more or less power during a charging session, the telemetered LSK data can also comprise an instruction to the charger 100 to increase or decrease the power of the magnetic field 145. In either case, adjusting the power of the magnetic field 145 may involve use of a different function DCx at the power module 214. This isn’t however strictly necessary, as the charger 100 can adjust the power of the magnetic field 145 without use of these functions.

In place of, or in addition to, use of LSK telemetry, the charger 100 can include short-range RF telemetry means for communicating with the IMD 10. Thus, the charger 100 and IMD 10 could both include short-range RF antennas and associated modulation/demodulation chip sets (not shown) to allow communications using far-field electromagnetic waves. Such communications could occur using the well-known Bluetooth standard for example. As such, the IMD 10 can provide telemetry (the EoC signal, other charging parameters or control signals) to the charger 100 using such means.

Although charger 100 is disclosed as being useful to wirelessly charger an IMD 10, one skilled will understand that charger 100 could be used to wirelessly charge any device, including those that are not implantable or that are not medical devices.

While the charger 100 is described above as having two sense coils—an outer and an inner sense coil 170o and 170i—this is not strictly necessary. Instead, one, or three or more, sense coils could be used.

For example, only one sense coil could be used in the charger 100. When using only one sense coil, LSK telemetry can be detected using only the signal induced on that sense coil using LSK demodulator 202. Thus, the amplitude of that single sense coil (A), or the phase angle of that sense coil relative to the drive signal X, or both (e.g., as cross correlated at 290) can be used to demodulate received LSK telemetry from the IMD 10. Likewise, the phase angle of that single sense coil relative to the drive signal X could be considered (and minimized) by the resonance module 216. Similarly, that phase angle could be used by the initial alignment algorithm 250 in the alignment module 210 to determine initial alignment. Lastly, the amplitude of that single sense coil (A) could be interpreted by the power module 214 to determine how to adjust the power of the magnetic field 145 (e.g., in accordance with a particular DC(A) function).

If three or more sense coils are used in the charger 100, preferred and most sensitive of the sense coils could be used in the charger to determine various conditions. Thus, the sense-coil-based signals assessed for determined LSK telemetry (at demodulator 202), frequency adjustment (at resonance module 216), alignment (at alignment module 210), or power adjustment (at power module 214), could be determined by assessing one or more of the most suitable induced sense coil signals.

Although examples of the charger 100 are described as being used in implantable stimulation devices systems such as a spinal cord stimulation system, the antennas, circuitries, and algorithms described herein may be used in other implantable medical device systems to charge other types of implantable medical devices. For example, in some implementations, the implantable medical device may include an electric pump that is configured to move fluid within the implantable medical device to inflate or deflate an inflatable member. For example, in some implementations, the implantable medical device may be an inflatable penile implant, or an inflatable artificial sphincter (such as an artificial urinary sphincter). In some cases, inflatable penile implants are used to help address erectile disfunction issues. In some cases, inflatable artificial sphincters are used to help address continence issues.

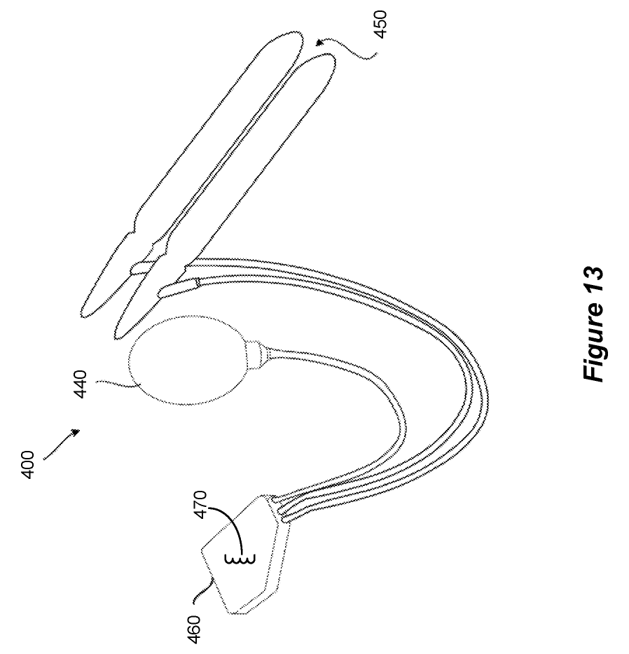

As illustrated in FIG. 13, an inflatable penile implant 400 (a type of implantable medical device, IMD) includes a fluid reservoir 440 configured to be placed within a pelvic region of a patient and one or more inflatable members 450 configured to be placed within a penis of the patient. The inflatable penile implant 400 also includes a housing 460 (including a case and a header) that is operatively coupled to the fluid reservoir 440 and to the inflatable member(s) 450. The housing 460 may house a power source (such as a rechargeable battery) and a pump or pumps (such as a piezo-electric pump or pumps) configured to move fluid to and from the inflatable member(s) 450 to place them in an inflated configuration or a deflated configuration. The housing 460 may also house an antenna or coil 470 analogous to secondary charging coil 36 (FIG. 3) to allow the inflatable penile implant 400 to receive the magnetic field 145 from the charger 100, thus providing power for the inflatable penile implant 400 and allowing its battery to be charged.

As discussed earlier, the various algorithms and modules described herein can be implemented as firmware or software, and such algorithms, firmware, or software may be embodied in a non-transitory computer readable media, such as a solid-state memory (e.g., control circuitry 124 in the charger 100), optical or magnetic disks, and the like. These media may be present outside of the charger 100, and stored in manners downloadable to the charger 100, such as on various Internet servers, portable or stationary disks, manufacturing computer systems, and the like.

lthough particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover equivalents that may fall within the spirit and scope of the present invention as defined by the claims.

What is claimed is:

1. An external charger for wirelessly providing energy to an implantable medical device (IMD), comprising:
a charging coil configured when energized to produce a magnetic field to wirelessly provide energy to the IMD during a charging session;
a first sense coil, wherein the first sense coil is configured to be induced by the magnetic field with a first induced signal;
a second sense coil, wherein the second sense coil is configured to be induced by the magnetic field with a second induced signal; and
control circuitry configured to determine a phase difference between the first and second induced signals.

2. The external charger of claim 1, wherein the control circuitry is further configured to determine an alignment between the charging coil and the IMD using the phase difference.

3. The external charger of claim 2, wherein the control circuitry is configured to determine the alignment by determining whether the charging coil and the IMD are aligned or misaligned using the phase difference.

4. The external charger of claim 3, wherein the control circuitry is configured to determine whether the charging coil and the IMD are aligned or misaligned by comparing the phase difference to a threshold.

5. The external charger of claim 4, wherein the control circuitry is configured to determine that the charging coil and the IMD are aligned when the phase difference is equal to or larger than the threshold, and to determine that the charging coil and the IMD are misaligned when the phase difference is less than the threshold.

6. The external charger of claim 3, further comprising a user interface comprising one or more user interface elements, wherein when the control circuitry determines that the charging coil and the IMD are misaligned, the control circuitry is further configured to enable one or more of the user interface elements.

7. The external charger of claim 6, wherein the one or more user interface elements comprise one or more of a visual indicator or a speaker.

8. The external charger of claim 2, wherein the control circuitry is configured to determine the alignment between the charging coil and the IMD using the phase difference while producing the magnetic field with a constant power and frequency.

9. The external charger of claim 1, further comprising a circuit board comprising the charging coil, the first sense coil, and the second sense coil.

10. The external charger of claim 9, wherein the charging coil comprises a wire winding affixed to a side of the circuit board.

11. The external charger of claim 9, wherein the first and second sense coils comprise one or more traces in the circuit board.

12. The external charger of claim 9, wherein the charging coil, the first sense coil, and the second sense coil are concentric.

13. The external charger of claim 12, wherein the first sense coil has a radius larger than a radius of the second sense coil.

14. The external charger of claim 13, wherein the charging coil has a radius larger than the radii of the first and second sense coils.

15. The external charger of claim 1, wherein the control circuitry is configured to determine the phase difference periodically during the charging session.

16. The external charger of claim 1, wherein the control circuitry is configured to use the phase difference to adjust a power of the magnetic field.

17. The external charger of claim 1, wherein the external charger comprises a first housing comprising the charging coil and the first and second sense coils, a second housing comprising the control circuitry, and a cable for passing signals between the first and second housings.

18. The external charger of claim 17, wherein the second housing further comprises an amplifier, wherein the amplifier is configured to energize the charging coil to produce the magnetic field, wherein the second housing further comprises a user interface.

19. A method for wirelessly providing energy to an implantable medical device (IMD), comprising:
energizing a charging coil in an external charger to produce a magnetic field to wirelessly provide energy to the IMD during a charging session;
inducing by the magnetic field a first induced signal on a first sense coil in the external charger;
inducing by the magnetic field a second induced signal on a second sense coil in the external charger; and
determining a phase difference between the first and second induced signals.

20. A computer readable medium comprising instructions executable in an external charger for wirelessly providing energy to an implantable medical device (IMD), wherein the instructions when executed are configured to:
energize a charging coil in the external charger to produce a magnetic field to wirelessly provide energy to the IMD during a charging session; and
determine a phase difference between a first induced signal induced on a first sense coil in the external charger and a second induced signal induced on a first sense coil in the external charger.
